SEQUENCE LISTING (23 sequences)

SEQ ID NO: 1 - DNA, 1293 bp, Rhodobacter sphaeroides
atggccctcgacgtgcagagcgatatcgtcgcctacgacgcgcccaagaaggacctctacgagatcggcgagatgccgcctctcggccatgtgccgaaggagatgtatgcttgggccatccggcgcgagcgtcatggcgagccggatcaggccatgcagatcgaggtggtcgagacgccctcgatcgacagccacgaggtgctcgttctcgtgatggcggcgggcgtgaactacaacggcatctgggccggcctcggcgtgcccgtctcgccgttcgacggtcacaagcagccctatcacatcgcgggctccgacgcgtcgggcatcgtctgggcggtgggcgacaaggtcaagcgctggaaggtgggcgacgaggtcgtgatccactgcaaccaggacgacggcgacgacgaggaatgcaacggcggcgacccgatgttctcgcccacccagcggatctggggctacgagacgccggacggctccttcgcccagttcacccgcgtgcaggcgcagcagctgatgaagcgtccgaagcacctgacctgggaagaggcggcctgctacacgctgaccctcgccaccgcctaccggatgctcttcggccacaagccgcacgacctgaagccggggcagaacgtgctggtctggggcgcctcgggcggcctcggctcctacgcgatccagctcatcaacacggcgggcgccaatgccatcggcgtcatctcagaggaagacaagcgcgacttcgtcatggggctgggcgccaagggcgtcatcaaccgcaaggacttcaagtgctggggccagctgcccaaggtgaactcgcccgaatataacgagtggctgaaggaggcgcgcaagttcggcaaggccatctgggacatcaccggcaagggcatcaacgtcgacatggtgttcgaacatccgggcgaggcgaccttcccggtctcgtcgctggtggtgaagaagggcggcatggtcgtgatctgcgcgggcaccaccggcttcaactgcaccttcgacgtccgctacatgtggatgcaccagaagcgcctgcagggcagccatttcgccaacctcaagcaggcctccgcggccaaccagctgatgatcgagcgccgcctcgatccctgcatgtccgaggtcttcccctgggccgagatcccggctgcccatacgaagatgtataagaaccagcacaagcccggcaacatggcggtgctggtgcaggccccgcgcacggggttgcgcaccttcgccgacgtgctcgaggccggccgcaaggcctga

SEQ ID NO: 2 - protein, 430 aa, Rhodobacter sphaeroides
MALDVQSDIVAYDAPKKDLYEIGEMPPLGHVPKEMYAWAIRRERHGEPDQAMQIEVVETPSIDSHEVLVLVMAAGVNYNGIWAGLGVPVSPFDGHKQPYHIAGSDASGIVWAVGDKVKRWKVGDEVVIHCNQDDGDDEECNGGDPMFSPTQRIWGYETPDGSFAQFTRVQAQQLMKRPKHLTWEEAACYTLTLATAYRMLFGHKPHDLKPGQNVLVWGASGGLGSYAIQLINTAGANAIGVISEEDKRDFVMGLGAKGVINRKDFKCWGQLPKVNSPEYNEWLKEARKFGKAIWDITGKGINVDMVFEHPGEATFPVSSLVVKKGGMVVICAGTTGFNCTFDVRYMWMHQKRLQGSHFANLKQASAANQLMIERRLDPCMSEVFPWAEIPAAHTKMYKNQHKPGNMAVLVQAPRTGLRTFADVLEAGRKA

SEQ ID NO: 3 - DNA, 1071 bp, Sulfolobus tokodaii
atgaggagaacattaaaagccgcaatattaggtgctactggtttagtaggaatcgaatacgtaagaatgctatcaaatcatccttatattaaaccagcatatttagctggaaaaggttcagtgggtaaaccgtatggtgaggtagtaagatggcaaacagtaggacaagttcctaaggaaatagctgatatggaaataaaaccaactgatcctaagttaatggatgatgtagacataatattttctccattacctcaaggtgctgctggcccagtagaagaacaatttgcaaaagaaggattccctgtgattagtaattcaccagatcatagatttgatcctgatgttcccttattggttcctgaactaaatcctcatactattagcttaattgatgagcaaagaaaaagaagagaatggaaaggatttatagtaactacaccactatgcacagcccagggtgcagcaataccattaggtgctatatttaaagattataagatggatggagcatttataactactattcaatcgctatctggtgccggttatccaggaataccatcattagatgtagtagataatatcttgcctttaggtgatggatacgatgccaagacgataaaagagatcttcagaattttaagcgaagttaagagaaatgtagatgaacctaaattagaagatgtaagcttagcagcaacaactcatagaatagctactatacatggtcattatgaagtactatatgtatcgttcaaagaggaaactgctgctgaaaaagttaaggagactttagaaaactttagaggggaaccacaagatctaaaattaccaactgcaccttcaaagccaattatcgttatgaatgaggatacaagacctcaagtctattttgatagatgggctggggatattccaggaatgagtgtagttgtaggtagattaaagcaagtgaataagagaatgataaggttagtatcattaattcataacacggtcagaggagccgcaggaggaggtatattagcagctgaattacttgtcgaaaaaggatatattgaaaagtaa

SEQ ID NO: 4 - protein, 356 aa, Sulfolobus tokodaii
MRRTLKAAILGATGLVGIEYVRMLSNHPYIKPAYLAGKGSVGKPYGEVVRWQTVGQVPKEIADMEIKPTDPKLMDDVDIIFSPLPQGAAGPVEEQFAKEGFPVISNSPDHRFDPDVPLLVPELNPHTISLIDEQRKRREWKGFIVTTPLCTAQGAAIPLGAIFKDYKMDGAFITTIQSLSGAGYPGIPSLDVVDNILPLGDGYDAKTIKEIFRILSEVKRNVDEPKLEDVSLAATTHRIATIHGHYEVLYVSFKEETAAEKVKETLENFRGEPQDLKLPTAPSKPIIVMNEDTRPQVYFDRWAGDIPGMSVVVGRLKQVNKRMIRLVSLIHNTVRGAAGGGILAAELLVEKGYIEK

SEQ ID NO: 5 - DNA, 3967 bp, Artificial Sequence
  Description of Artificial Sequence Synthetic vector polynucleotide
atgcgccgcaccctgaaggcagcaatcctgggcgccaccggcctggtgggcatcgaatacgtgcgcatgctgtccaaccacccatacatcaagccagcatacctggccggcaagggctccgttggcaagccatacggcgaagtggtgcgctggcagaccgtgggccaggtgccaaaggaaatcgcagatatggaaatcaagccaaccgatccaaagctgatggatgatgtggatatcatcttctccccactgccacagggtgcagcaggcccagtggaagaacagttcgcaaaggaaggcttcccagtgatctccaactccccagatcaccgcttcgatccagatgtgccactgctggtgccagaactcaacccacacaccatctccctgatcgatgaacagcgcaagcgccgcgaatggaagggcttcatcgtgaccaccccactgtgcaccgcacagggcgcagcaatcccactgggcgcaatcttcaaggattacaagatggatggcgcattcatcaccaccatccagtccctgtccggcgcaggctacccaggtatcccatccctggatgtggtggataacatcctgccactgggcgatggctacgatgcaaagaccatcaaggaaatcttccgcatcctgtccgaagtgaagcgcaacgtggatgaaccaaagctggaagatgtgtccctggccgcaaccacccaccgcatcgcaaccatccacggccactacgaagtgctgtacgtgtccttcaaggaagaaaccgcagcagaaaaggtgaaggaaaccctggaaaacttccgcggcgaaccacaggatctgaagctgccaaccgcaccatccaagccaatcatcgtgatgaacgaagatacccgcccacaggtgtacttcgatcgctgggcaggcgatatcccaggcatgtccgtggtggtgggccgcctgaagcaggtgaacaagcgcatgatccgcctggtgtccctgatccacaacaccgttcgcggcgcagcaggtggtggtatcctggccgcagaactcctggtggaaaagggctacatcgaaaagtaagaattccgcgagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggctatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgcggatccaaggagatatagat

SEQ ID NO: 6 - DNA, 3766 bp, Artificial Sequence
  Description of Artificial Sequence Synthetic vector polynucleotide
atggaaaaggtggcattcatcggcctgggcgcaatgggctacccaatggcaggtcacctggctcgccgcttcccaaccctggtgtggaaccgcaccttcgaaaaggcactgcgccaccaggaagagttcggctccgaagcagtgccactggaacgcgtggctgaagcacgcgtgatcttcacctgcctgccaaccacccgcgaagtgtacgaagtggcagaagcactgtacccatacctgcgcgaaggcacctactgggtggatgcaacctccggcgaaccagaagcatcccgccgcctggctgaacgcctgcgcgaaaagggcgtgacctacctggatgcaccagtgtccggtggcacctccggtgcagaagcaggcaccctgaccgttatgctgggcggtccagaagaagcagtcgaacgcgtccgcccattcctggcctacgcaaagaaggtggtccacgtcggcccagttggtgcaggccacgcagtgaaggcaatcaacaacgcactgctggccgtgaacctgtgggcagcaggcgaaggtctgctggccctggtgaagcagggcgtgtccgcagaaaaggccctggaagtgatcaacgcatcctccggccgctccaacgcaaccgaaaacctgatcccacagcgcgttctgacccgcgcattcccaaagaccttcgcactgggcctgctggtgaaggatctgggcatcgcaatgggcgtgctggatggcgaaaaggcaccatccccactgctgcgcctggctcgcgaagtctacgagatggcaaagcgcgaactcggcccagatgcagatcacgtggaagcactgcgcctgctcgaacgctggggcggtgtggaaatccgctaagaattccgcgagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggctatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgcggatccaaggagatatagat

SEQ ID NO: 7 - DNA, 30 bp, Artificial Sequence
  Description of Artificial Sequence Synthetic oligonucleotide
ggaggcaaccatggccctcgacgtgcagag

SEQ ID NO: 8 - DNA, 32 bp, Artificial Sequence
  Description of Artificial Sequence Synthetic oligonucleotide
gagacttgcggatccctccgatcaggccttgc

SEQ ID NO: 9 - DNA, 47 bp, Artificial Sequence
  Description of Artificial Sequence Synthetic oligonucleotide
tgaagatcctaggaggtttaaacatatgccgttaatagccgggattg

SEQ ID NO: 10 - DNA, 33 bp, Artificial Sequence
  Description of Artificial Sequence Synthetic oligonucleotide
tatatagtcgacttaattcgcctgaccggccag

SEQ ID NO: 11 - DNA, 29 bp, Artificial Sequence
  Description of Artificial Sequence Synthetic oligonucleotide
tatataacatgtcgctttcaccgccaggc

SEQ ID NO: 12 - DNA, 50 bp, Artificial Sequence
  Description of Artificial Sequence Synthetic oligonucleotide
catatgtttaaacctcctaggatcttcagtttctctcacttaacggcagg

SEQ ID NO: 13 - DNA, 29 bp, Artificial Sequence
  Description of Artificial Sequence Synthetic oligonucleotide
tatataccatggcgctttcaccgccaggc

SEQ ID NO: 14 - DNA, 40 bp, Artificial Sequence
  Description of Artificial Sequence Synthetic oligonucleotide
tatatacatatgaaaagatcaaaacgatttgcagtactgg

SEQ ID NO: 15 - DNA, 37 bp, Artificial Sequence
  Description of Artificial Sequence Synthetic oligonucleotide
tatatagtcgacttagcttcctttacgcagcttatgc

SEQ ID NO: 16 - DNA, 34 bp, Artificial Sequence
  Description of Artificial Sequence Synthetic oligonucleotide
aaaacatatgaattttcatcatctggcttactgg

SEQ ID NO: 17 - DNA, 49 bp, Artificial Sequence
  Description of Artificial Sequence Synthetic oligonucleotide
aaaacatatgtatatttccttctttcaggcctccaggcttatccagatg

SEQ ID NO: 18 - DNA, 30 bp, Artificial Sequence
  Description of Artificial Sequence Synthetic oligonucleotide
aaaacatatgatcgacactgcgccccttgc

SEQ ID NO: 19 - DNA, 28 bp, Artificial Sequence
  Description of Artificial Sequence Synthetic oligonucleotide
aagacgtcctaccgctcgccggccgtcc

SEQ ID NO: 20 - DNA, 23 bp, Artificial Sequence
  Description of Artificial Sequence Synthetic oligonucleotide
aaccatgggccgcaccctgaagg

SEQ ID NO: 21 - DNA, 33 bp, Artificial Sequence
  Description of Artificial Sequence Synthetic oligonucleotide
aaggatccttacttttcgatgtagcccttttcc

SEQ ID NO: 22 - DNA, 29 bp, Artificial Sequence
  Description of Artificial Sequence Synthetic oligonucleotide
aaaacatatggaaaaggtggcattcatcg

SEQ ID NO: 23 - DNA, 30 bp, Artificial Sequence
  Description of Artificial Sequence Synthetic oligonucleotide
aaaagatctttagcggatttccacaccgcc

(19) United States
(12) Patent Application Publication
(10) Pub. No.: US 2010/0190224 A1
(43) Pub. Date: Jul. 29, 2010

(54) ENZYME FOR THE PRODUCTION OF METHYLMALONYL-COENZYME A OR ETHYLMALONYL-COENZYME A AND USE THEREOF

(75) Inventors: Markus Poetter, Muenster (DE); Achim Marx, Gelnhausen (DE); Georg Fuchs, Heitersheim (DE); Birgit Alber, Stuttgart (DE); Tobias Juergen Erb, Freiburg i. Breisgau (DE)

Correspondence Address: OBLON, SPIVAK, MCCLELLAND MAIER & NEUSTADT, L.L.P., 1940 DUKE STREET, ALEXANDRIA, VA, 22314, US

(73) Assignees: EVONIK DEGUSSA Gmbh, Essen (DE); ALBERT-LUDWIG-UNIVERSITAET FREIBURG, Freiburg (DE)

(21) Appl. No.: 12/593,090

(22) PCT Filed: Mar. 27, 2008
(86) PCT No.: PCT/EP08/53655

§ 371 Date: Mar. 26, 2010

(30) Foreign Application Priority Data
Mar. 29, 2007 (DE) 10 2007 015 583.4

(51) Int. Cl. C12P 7/42; C12N 15/74; C12N 9/00; C12N 1/21; C12N 15/63; C07H 21/04

(52) U.S. Cl. 435/146; 435/471; 435/183; 435/252.3; 435/320.1; 536/23.2; 536/23.7; 536/24.5

(57) ABSTRACT

The present invention relates to an isolated DNA, which is selected from the following sequences:
a) a sequence according to SEQ ID No. 01,
b) an intron-free sequence that is derived from a sequence according to a) and encodes the same protein or peptide as the sequence according to SEQ ID No. 01,
c) a sequence that encodes a protein or peptide, which comprises the amino acid sequence according to SEQ ID No. 02,
d) a sequence that is at least 80% identical to a sequence according to a) to c),
e) a sequence that hybridizes with the antisense strand of a sequence according to one of the groups a) to d) or would hybridize taking into account degeneration of the genetic code,
f) a derivative of a sequence according to one of the groups a) to e) obtained by substitution, addition, inversion and/or deletion of one or more bases,
g) a sequence that corresponds to SEQ ID No. 01 within the degeneration of the genetic code,
h) a sequence with neutral sense mutations of SEQ ID No. 01, and
i) a sequence complementary to a sequence according to one of the groups a) to h).

The invention further relates to a vector, the use of this vector for transformation of a cell, a transformed cell, a polypeptide, cells genetically engineered relative to their wild type, a method of production of a genetically engineered cell, the genetically engineered cell obtainable by this method, the use of this cell and a method of production of 3-hydroxyisobutyric acid or of a derivative thereof.

The present invention relates to an isolated DNA, a vector, the use of said vector for the transformation of a cell, a transformed cell, a polypeptide, cells genetically engineered relative to their wild type, a method of production of a genetically engineered cell, the genetically engineered cell obtainable by said method, the use of said cell and a method of production of organic C₃ and/or C₄ compounds.

Organic C₃ and C₄ compounds, for example 3-hydroxypropionic acid or 3-hydroxyisobutyric acid (3-HIB), are important chemical precursors and are used for example for the production of medicinal active substances or as components in the production of biodegradable polymers. Thus, 3-hydroxyisobutyric acid is suitable for example as a precursor in the synthesis of epicaptopril, an angiotensin-converting-enzyme (ACE) inhibitor, which is used inter alia for the treatment of hypertension. 3-Hydroxyisobutyric acid can also be converted to methacrylic acid by dehydration. 3-Hydroxypropionic acid is used for example for the production of acrylic acid by dehydration, for the production of malonic acid by oxidation or for the production of 1,3-propanediol by reduction.

Fermentation methods of production of 3-hydroxyisobutyric acid or 3-hydroxypropionic acid are known from the state of the art. Thus, U.S. Pat. No. 4,618,583 describes a method of production of 3-hydroxyisobutyric acid, in which substrates selected from the group comprising isobutyric acid, methacrylic acid, isobutyryl chloride, the methyl ester of isobutyryl chloride, methyl ester of methacrylic acid, the ethyl ester of methacrylic acid, isobutyl alcohol, esters of isobutyl alcohol, isobutylamine, isobutylaldehyde, isobutylamide or mixtures thereof are converted using microorganisms of the genera Pseudomonas aeruginosa or Protaminobacter alboflavus to 3-hydroxyisobutyric acid. WO-A-03/62173, WO-A-02/42418 and WO-A-01/16346 describe the production of 3-hydroxypropionic acid from carbohydrates or glycerol by recombinant cells, employing various metabolic pathways.

The disadvantage of the fermentation methods described above for the production of 3-hydroxyisobutyric acid or 3-hydroxypropionic acid is that, among other things, the amount of target product formed in the fermentation solution is too small for this fermentation solution to be used as starting material for large-scale production of further products based on 3-hydroxyisobutyric acid or 3-hydroxypropionic acid.

Furthermore, in the methods known from the state of the art for the production of 3-hydroxypropionic acid or 3-hydroxyisobutyric acid, C₆ compounds such as glucose or alternatively C₃ compounds such as pyruvate, phosphoenolpyruvate or glycerol are always used, so that these methods are essentially limited to the use of carbohydrates or of glycerol as carbon sources. There is, however, increasing interest in producing organic C₃ or C₄ compounds also from C₁ or C₂ carbon sources, such as carbon dioxide, methane, methanol or ethanol. However, such a synthesis of C₃ or C₄ compounds from C₁ or C₂ carbon sources is not possible in an economically meaningful way by the methods known from the state of the art.

The present invention was based on the problem of overcoming the disadvantages arising from the state of the art.

In particular, the present invention was based on the problem of providing a nucleic acid sequence that encodes an enzyme which, if it is overexpressed in a suitable cell, enables this cell to produce organic C₃ and/or C₄ compounds, in particular 3-hydroxyisobutyric acid or derivatives thereof, in amounts as large as possible from suitable carbon sources, in particular from C₁ or C₂ carbon sources.

The present invention was also based on the problem of providing an enzyme which, in comparison with the enzymes known from the state of the art, on overexpression of this enzyme in the cell, decisively improves the ability of the cell to form 3-hydroxyisobutyric acid or derivatives thereof.

Furthermore, the present invention was based on the problem of providing a cell that is able to produce, from suitable carbon sources, in particular also from C₁ or C₂ carbon sources, methylmalonyl-coenzyme A or ethylmalonyl-coenzyme A as possible intermediates in a method of production of 3-hydroxyisobutyric acid or derivatives thereof, or alternatively 3-hydroxyisobutyric acid or derivatives thereof directly, even better, in particular even more efficiently than the cells described in the state of the art.

An isolated DNA, which is selected from the following sequences, makes a contribution to solution of the aforementioned problems:
a) a sequence according to SEQ ID No. 01,
b) an intron-free sequence, which is derived from a sequence according to a) and which encodes the same protein or peptide as the sequence according to SEQ ID No. 01,
c) a sequence that encodes a protein or peptide, which is covered by the amino acid sequence according to SEQ ID No. 02,
d) a sequence that is identical to a sequence according to one of the groups a) to c), especially preferably according to group a), to at least 80%, especially preferably to at least 90%, even more preferably to at least 95% and most preferably to at least 99%, and this sequence preferably encodes a protein or peptide that is capable of converting crotonyl-coenzyme A to ethylmalonyl-coenzyme A and optionally also acrylyl-coenzyme A to methylmalonyl-coenzyme A,
e) a sequence that hybridizes with the antisense strand of a sequence according to one of the groups a) to d), especially preferably according to group a), or would hybridize, taking into account degeneration of the genetic code, and said sequence preferably encodes a protein or peptide that is capable of converting crotonyl-coenzyme A to ethylmalonyl-coenzyme A and optionally also acrylyl-coenzyme A to methylmalonyl-coenzyme A,
f) a derivative of a sequence according to one of the groups a) to e), especially preferably according to group a) obtained by substitution, addition, inversion and/or deletion of at least one base, preferably of at least 2 bases, more preferably of at least 5 bases and most preferably at least 10 bases, however, preferably of not more than 100 bases, especially preferably of not more than 50 bases and most preferably of not more than 25 bases, and said derivative preferably encodes a protein or peptide that is capable of converting crotonyl-coenzyme A to ethylmalonyl-coenzyme A and optionally also acrylyl-coenzyme A to methylmalonyl-coenzyme A,
g) a sequence that corresponds to SEQ ID No. 01 within the degeneration of the genetic code,
h) a sequence with neutral sense mutations of SEQ ID No. 01, and
i) a sequence complementary to a sequence according to one of the groups a) to h), especially preferably according to group a).

It was found, surprisingly, that a DNA sequence isolated from bacteria of the strain Rhodobacter sphaeroides, with a DNA sequence according to SEQ ID No. 01, encodes a polypeptide (SEQ ID No. 02) that is capable of converting both crotonyl-coenzyme A and acrylyl-coenzyme A to the corresponding alkylmalonyl-coenzyme A compounds (ethylmalonyl-coenzyme A or methylmalonyl-coenzyme A). Since ethylmalonyl-coenzyme A and methylmalonyl-coenzyme A are natural metabolic products, which for example are formed in the degradation of valine, of leucine or of isoleucine or in the metabolism of propanoate or in the ethylmalonyl-coenzyme A cycle of certain bacteria and because these alkylmalonyl-coenzyme A compounds can be further reduced via the corresponding semialdehydes in the course of the aforementioned metabolic pathways to the corresponding 3-hydroxyalkanoates, the isolated DNA according to the invention can be used for producing recombinant bacteria that are capable of directly forming large amounts of 3-hydroxyisobutyric acid (and optionally also 3-hydroxypropionic acid). If the cells were in addition capable of polymerizing the 3-hydroxyalkanoates formed, producing polyhydroxyalkanoates, this DNA would additionally be suitable for producing recombinant bacteria that can produce polyhydroxyalkanoates based on 3-hydroxyisobutyric acid (or optionally also based on 3-hydroxypropionic acid).

The “nucleotide identity” as well as the “amino acid identity” in the sense of the present invention are determined by known methods. Generally special computer programs are used with algorithms taking into account special requirements. Preferred methods of determination of identity firstly produce the greatest agreement between the sequences to be compared. Computer programs for determining identity comprise, but are not restricted to, the GCG software package, including
GAP (Deveroy, J. et al., Nucleic Acid Research 12 (1984), page 387, Genetics Computer Group University of Wisconsin, Medicine (Wi), and
BLASTP, BLAST and FASTA (Altschul, S. et al., Journal of Molecular Biology 215 (1990), pages 403-410. The BLAST program can be obtained from the National Center for Biotechnology Information (NCBI) and from other sources (BLAST Manual, Altschul S. et al., NCBI NLM NIH Bethesda ND 22894; Altschul S. et al., as above).

A person skilled in the art is aware that various computer programs are available for the calculation of the similarity or identity between two nucleotide or amino acid sequences. Thus, the percentage identity between two amino acid sequences can be determined e.g. using the algorithm of Needleman and Wunsch (J. Mol. Biol. (48): 444-453 (1970)), in the GAP program in the GCG software package (obtainable from http://www.gcg.com), using either a Blossom 62 matrix or a PAM250 matrix, with a gap weight of 16, 14, 12, 10, 8, 6, or 4 and a length weight of 1, 2, 3, 4, 5, or 6. A person skilled in the art will recognize that the use of different parameters will lead to slightly different results, but the percentage identity between two amino acid sequences will not be significantly different overall. Usually the Blossom 62 matrix is used with the defaults (gap weight: 12, length weight: 1).

An identity of 80% according to the aforementioned algorithm means, in the context of the present invention, 80% identity. The same applies to higher identities.

The feature “sequence that hybridizes with the antisense strand of a sequence according to one of the groups a) to d), especially preferably according to group a), or would hybridize taking into account degeneration of the genetic code” according to alternative e) indicates a sequence that hybridizes under preferably stringent conditions with the antisense strand of a sequence according to one of the groups a) to d), especially preferably according to group a), or would hybridize taking into account degeneration of the genetic code. For example, the hybridizations can be carried out at 68° C. in 2×SSC or according to the protocol of the Dioxygenin-Labelling-Kit of the company Boehringer (Mannheim). Preferred hybridization conditions are e.g. incubation at 65° C. overnight in 7% SDS, 1% BSA, 1 mM EDTA, 250 mM sodium phosphate buffer (pH 7.2) and then washing at 65° C. with 2×SSC; 0.1% SDS.

The derivatives of the isolated DNA according to the invention, which according to alternative f) can be obtained by substitution, addition, inversion and/or deletion of one or more bases of a sequence according to one of the groups a) to e), include in particular such sequences that can lead, in the protein that they encode, to conservative amino acid exchanges, e.g. exchange of glycine for alanine or of aspartic acid for glutamic acid. These function-neutral mutations are called sense mutations, and do not result in any fundamental change in activity of the polypeptide. Moreover, it is known that changes at the N- and/or C-terminus of a polypeptide do not greatly affect its function or can even stabilize it, and accordingly DNA sequences in which bases are added at the 3′-end or at the 5′-end of the sequence with SEQ ID No. 01 are also covered by the present invention. A person skilled in the art will find information on this in, among others, Ben-Bassat et al. (Journal of Bacteriology 169:751-757 (1987)), O'Regan et al. (Gene 77:237-251 (1989)), Sahin-Toth et al. (Protein Sciences 3:240-247 (1994)), Hochuli et al. (Bio/Technology 6:1321-1325 (1988)) and in known textbooks of genetics and molecular biology.

For isolation of the DNA according to the invention, first the chromosomal DNA was isolated from Rhodobacter sphaeroides according to F. M. Ausubel et al., “Current Protocols in Molecular Biology”, John Wiley and Sons, New York, 1987. A homologous nucleic acid sequence was identified in this DNA, which encodes a protein that is 78% identical to the crotonyl-coenzyme A-reductase gene (ccr gene) from Methylobacterium extorquens, 41% identical to the ccr gene from S. collinus and 39% identical to the ccr gene from S. coelicolor. Then, using two synthetic polynucleotides, as described in more detail below in Example 1, the complete ccr gene from Rhodobacter sphaeroides was amplified by PCR.

A further contribution to solution of the problems stated at the beginning is provided by a vector, preferably an expression vector, comprising a DNA with a sequence according to one of the groups a) to h), as defined above. As vectors, consideration may be given to all vectors known by a person skilled in the art that are usually employed for inserting DNA into a host cell. Preferred vectors are selected from the group comprising plasmids, such as the E. coli plasmids pTE13, pTrc99A, pBR345 and pBR322, viruses, such as bacteriophages, adenoviruses, vaccinia viruses, baculoviruses, measles viruses and retroviruses, cosmids or YACs, plasmids being most preferred as vectors.

According to a preferred embodiment of the vector according to the invention, the DNA with a sequence according to one of the groups a) to h) is under the control of a controllable promoter, which is suitable for expression of the polypeptide encoded by these DNA sequences in the cell of a microorganism, preferably a bacterial, yeast or fungal cell, especially preferably a bacterial cell, most preferably an E. coli cell. Examples of such promoters are the trp-promoter or the tac-promoter.

The vector according to the invention should include, in addition to a promoter, preferably a ribosome-binding site and a terminator. It is especially preferable for the DNA according to the invention to be incorporated in an expression cassette of the vector comprising the promoter, the ribosome-binding site and the terminator. As well as the structural elements stated above, the vector can further comprise selection genes known by a person skilled in the art.

A further contribution to solution of the problems stated at the beginning is provided by the use of the vector described above for the transformation of a cell and the cell obtained by transformation with this vector. The cells that can be transformed with the vector according to the invention can be prokaryotes or eukaryotes. They can be mammalian cells (such as human cells), plant cells or microorganisms such as yeasts, fungi or bacteria, with microorganisms being especially preferred and bacteria and yeasts being most preferred.

Especially suitable as bacteria, yeasts or fungi are those bacteria, yeasts or fungi that have been deposited in the Deutsche Sammlung von Mikroorganismen and Zellkulturen GmbH (DSMZ), Braunschweig, Germany, as bacterial, yeast or fungal strains. Bacteria that are suitable according to the invention belong to the genera that are listed under

http://www.dsmz.de/species/bacteria.htm

Yeasts that are suitable according to the invention belong to those genera that are listed under

http://www.dsmz.de/species/yeasts.htm

Fungi that are suitable according to the invention are those listed under

http://www.dsmz.de/species/fungi.htm

In particular, according to an especially preferred embodiment of the use according to the invention of the vector described above, it may prove advantageous to transform a methanotrophic or methylotrophic microorganism, preferably a methylotrophic microorganism, with the vector. Methylotrophic microorganisms are able to utilize C₁ carbon compounds, for example methane, methanol or methylamines. As examples, we may mention bacteria of the genus Methylobacterium, such as Methylobacterium adhaesivum, Methylobacterium aminovorans, Methylobacterium aquaticum, Methylobacterium dichloromethanicum, Methylobacterium extorquens, Methylobacterium fujisawaense, Methylobacterium hispanicum, Methylobacterium isbiliense, Methylobacterium lusitanum, Methylobacterium mesophilicum, Pseudomonas mesophilica, Methylobacterium organophilum, Methylobacterium podarium, Methylobacterium radiotolerans, Pseudomonas radiora, Methylobacterium rhodesianum, Methylobacterium rhodinum, Methylobacterium sp., Methylobacterium suomiense, Methylobacterium thiocyanatum, Methylobacterium variabile or Methylobacterium zatmanii, bacteria of the genus Rhodobacter, Rhodobacter adriaticus, Rhodobacter adriaticus, Rhodopseudomonas adriatica, Rhodobacter blasticus, Rhodobacter capsulatus, Rhodobacter euryhalinus, Rhodovulum euryhalinum, Rhodobacter euryhalinus, Rhodobacter indicus, Rhodobacter sp., Rhodobacter sphaeroides, Rhodqpseudomonas sphaeroides, Rhodobacter sulfidophilus or Rhodobacter veldkampii, bacteria of the genus Streptomyces, such as Streptomyces coelicolor.

Cells especially preferred according to the invention are those of the genera Corynebacterium, Brevibacterium, Bacillus, Lactobacillus, Lactococcus, Candida, Pichia, Kluveromyces, Saccharomyces, Escherichia, Zymomonas, Yarrowia, Methylobacterium, Ralstonia, Rhodobacter and Clostridium, with Methylobacterium and Rhodobacter being especially preferred.

A contribution to solution of the problems mentioned at the beginning is also provided by a polypeptide that has the amino acid sequence with SEQ ID No. 02 or has an amino acid sequence that possesses identity of at least 50%, preferably at least 55%, even more preferably at least 60%, even more preferably at least 65% and most preferably at least 70% to the amino acid sequence according to SEQ ID No. 02. The polypeptide is an enzyme that is capable of converting both crotonyl-coenzyme A and acrylyl-coenzyme A to the corresponding alkylmalonyl-coenzyme A compounds (ethylmalonyl-coenzyme A or methylmalonyl-coenzyme A). Said polypeptide can be obtained for example by a synthetic route, starting from the DNA sequence with SEQ ID No. 01 or by transformation of a suitable cell with a suitable vector comprising this nucleic acid sequence, expression of the protein encoded by this nucleic acid sequence in the cell, lysis of the cell to obtain a cellular extract and subsequent purification of the enzyme by purification techniques known by a person skilled in the art, for example by HPLC or other chromatographic methods.

A contribution to solution of the problems mentioned at the beginning is also provided by a cell that has been genetically engineered relative to its wild type to be capable, in comparison with its wild type, of forming more ethylmalonyl-coenzyme A or more methylmalonyl-coenzyme A, preferably as intermediates in a method of production of 3-hydroxyisobutyric acid or derivatives thereof, or of forming more 3-hydroxyisobutyric acid or derivatives thereof directly. It is in particular preferable for the cell according to the invention to form, in a specified time interval, preferably within 2 hours, more preferably within 8 hours and most preferably within 24 hours, at least twice, especially preferably at least 10-fold, more preferably at least 100-fold, even more preferably at least 1000-fold and most preferably at least 10 000-fold more 3-hydroxyisobutyric acid or derivatives thereof than the wild-type cell. The increase in product formation can be determined for example by cultivating the cell according to the invention and the wild-type cell each separately under identical conditions (same cell density, same nutrient medium, same culture conditions) for a specified time interval in a suitable culture medium and then determining the amount of target product (3-hydroxyisobutyric acid or derivatives thereof) in the culture medium.

The term “3-hydroxyisobutyric acid”, as used here, always describes the corresponding C₄-carboxylic acid in the form in which it is present after formation by the corresponding microorganisms in relation to the pH value. The term thus always comprises the pure acid form (3-hydroxyisobutyric acid), the pure base form (3-hydroxyisobutyrate) and mixtures of the protonated and deprotonated form of the acid. Moreover, the term “3-hydroxyisobutyric acid” basically comprises both the (R) and the (S) stereoisomer, with the (S) stereoisomer being especially preferred. Also in connection with the alkylmalonyl-coenzyme A intermediates, the designation “methylmalonyl-coenzyme A” and “ethylmalonyl-coenzyme A” always comprises both the (R) and the (S) stereoisomer.

The term “derivative of 3-hydroxyisobutyric acid” preferably means polyhydroxyalkanoates, which are based on 3-hydroxyisobutyric acid as monomer.

The formulation “capable of forming more ethylmalonyl-coenzyme A and/or methylmalonyl-coenzyme A” and the formulation “capable of forming more 3-hydroxyisobutyric acid or derivatives thereof” also include the case when the wild type of the genetically engineered cell was not able to form any ethylmalonyl-coenzyme A, any methylmalonyl-coenzyme A, any 3-hydroxyisobutyric acid or any derivatives of 3-hydroxyisobutyric acid at all, or at least no detectable amounts of these compounds, and it is only after genetic manipulation that detectable amounts of these components can be formed.

A “wild type” of a cell preferably designates a cell whose genome is in a state such as arose naturally through evolution. The term is used both for the complete cell and for individual genes. The term “wild type” therefore in particular does not include such cells or such genes whose gene sequences have been altered at least partially by human intervention by recombinant techniques.

As cells, those cells are preferred that have already been mentioned as preferred cells in connection with the use of the vector according to the invention for the transformation of a cell.

The cell according to the invention preferably displays, in comparison with its wild type, an increased activity of the enzyme E₁, which is capable of catalysing the conversion of crotonyl-coenzyme A to ethylmalonyl-coenzyme A and of acrylyl-coenzyme A to methylmalonyl-coenzyme A. This enzyme E₁ is encoded by a DNA sequence according to one of the alternatives a) to h) or has the polypeptide sequence according to the invention with SEQ ID No. 02 or has an amino acid sequence displaying identity of at least 50%, preferably at least 55%, more preferably at least 60%, even more preferably at least 65% and most preferably at least 70% to the amino acid sequence according to SEQ ID No. 02, but is able to convert crotonyl-coenzyme A to ethylmalonyl-coenzyme A and optionally also acrylyl-coenzyme A to methylmalonyl-coenzyme A. The DNA sequence according to the invention can moreover be integrated in the genome of the cell or can be present on a vector inside the cell.

The statements that now follow concerning increasing the enzyme activity in cells, apply both to increasing the activity of the enzyme E₁ and to all enzymes stated hereunder, whose activity can possibly be increased.

Basically, an increase in enzymatic activity can be achieved by increasing the copy number of the gene sequence or of the gene sequences that encode the enzyme, using a strong promoter or using a gene or allele that encodes a corresponding enzyme with increased activity and optionally combines these measures. Cells genetically engineered according to the invention are produced for example by transformation, transduction, conjugation or a combination of these methods with a vector that contains the desired gene, an allele of this gene or portions thereof and a vector that makes expression of the gene possible. Heterologous expression is achieved in particular by integrating the gene or the alleles into the chromosome of the cell or to an extrachromosomally replicating vector.

A review of the possibilities for increasing the enzyme activity in cells for the example of pyruvate carboxylase is given in DE-A-100 31 999, which is hereby introduced as reference and whose disclosures with respect to the possibilities for increasing the enzyme activity in cells forms part of the disclosure of the present invention.

Expression of the enzymes or genes stated above and all those stated hereunder is detected by means of 1- and 2-dimensional protein gel separation and subsequent optical identification of the protein concentration in the gel with appropriate evaluating software. When the increase in enzyme activity is based exclusively on an increase in expression of the corresponding gene, the increase in enzyme activity can be quantified simply by comparing the 1- or 2-dimensional protein separations between the wild-type cell and the genetically engineered cell. A usual method for preparation of the protein gels for example in the case of coryneform bacteria and for identifying the proteins is the procedure described by Hermann et al. (Electrophoresis, 22: 1712.23 (2001). The protein concentration can also be analysed by Western-blot hybridization with a specific antibody to the protein to be detected (Sambrook et al., Molecular Cloning: a laboratory manual, 2nd ed. Cold Spring Harbor Laboratory Press, Cold Spring Harbor, N.Y. USA, 1989) followed by optical evaluation with appropriate software for determination of the concentration (Lohaus and Meyer (1989) Biospectrum, 5: 32-39; Lottspeich (1999), Angewandte Chemie 111: 2630-2647). The activity of DNA-binding proteins can be measured by DNA-band-shift assays (also called gel retardation) (Wilson et al. (2001) Journal of Bacteriology, 183: 2151-2155). The effect of DNA-binding proteins on the expression of other genes can be detected by various well-described methods of reporter gene assay (Sambrook et al., Molecular Cloning: a laboratory manual, 2nd ed. Cold Spring Harbor Laboratory Press, Cold Spring Harbor, N.Y. USA, 1989). Intracellular enzymatic activities can be determined by various methods that have been described (Donahue et al. (2000) Journal of Bacteriology 182 (19): 5624-5627; Ray et al. (2000) Journal of Bacteriology 182 (8): 2277-2284; Freedberg et al. (1973) Journal of Bacteriology 115 (3): 816-823). If, in the subsequent account, no concrete methods are stated for determination of the activity of a particular enzyme, the increase in enzyme activity and also the decrease in enzyme activity are preferably determined by the methods described in Hermann et al., Electophoresis, 22: 1712-23 (2001), Lohaus et al., Biospektrum 5 32-39 (1998), Lottspeich, Angewandte Chemie 111: 2630-2647 (1999) and Wilson et al., Journal of Bacteriology 183: 2151-2155 (2001).

If the increase in enzyme activity is brought about by mutation of the endogenous gene, then such mutations can be produced either randomly by classical methods, such as by UV-irradiation or with chemicals that trigger mutations, or in a directed manner by gene technology methods such as deletion(s), insertion(s) and/or nucleotide exchange(s). These mutations lead to the production of genetically engineered cells. Especially preferred mutants of enzymes are in particular those enzymes that are no longer susceptible to feedback inhibition or whose susceptibility is at least reduced in comparison with the wild-type enzyme.

If the increase in enzyme activity is brought about by increasing the expression of an enzyme, then for example the copy number of the corresponding genes is increased or the promoter and regulatory region or the ribosome-binding site that is located upstream of the structural gene, is mutated. Expression cassettes that are inserted upstream of the structural gene act in the same way. By means of inducible promoters it is additionally possible to increase the expression at any point of time. Furthermore, the enzyme gene can also be assigned so-called “enhancers” as regulatory sequences, which also lead to increased gene expression through improved interaction between RNA-polymerase and DNA. Expression is also improved by measures for extending the life of the m-RNA. Furthermore, enzyme activity is also intensified by preventing degradation of the enzyme protein. The genes or gene constructs are then either present in plasmids with variable copy number or are integrated and amplified in the chromosome. Alternatively, moreover, overexpression of the genes in question can be achieved by changing the composition of the medium and the culture conditions. A person skilled in the art will find instructions on this in, among others, Martin et al. (Bio/Technology 5, 137-146 (1987)), in Guerrero et al. (Gene 138, 35-41 (1994)), Tsuchiya and Morinaga (Bio/Technology 6, 428-430 (1988)), in Eikmanns et al. (Gene 102, 93-98 (1991)), in EP-A-0 472 869, in U.S. Pat. No. 4,601,893, in Schwarzer and Pithier (Bio/Technology 9, 84-87 (1991), in Reinscheid et al. (Applied and Environmental Microbiology 60, 126-132 (1994)), in LaBarre et al. (Journal of Bacteriology 175, 1001-1007 (1993)), in WO-A-96/15246, in Malumbres et al. (Gene 134, 15-24 (1993), in JP-A-10-229891, in Jensen and Hammer (Biotechnology and Bioengineering 58, 191-195 (1998)) and in known textbooks of genetics and molecular biology. The measures described above lead, just like the mutations, to genetically engineered cells.

Episomal plasmids are used, for example, for increasing the expression of the respective genes. Suitable plasmids are, in particular, those that are replicated in coryneform bacteria. Numerous known plasmid vectors, for example pZ1 (Menkel et al., Applied and Environmental Microbiology 64: 549-554 (1989)), pEKEx1 (Eikmanns et al., Gene 107: 69-74 (1991)) or pHS2-1 (Sonnen et al., Gene 107: 69-74 (1991)) are based on the cryptic plasmids pHM1519, pBL1 or pGA1. Other plasmid vectors, for example those based on pCG4 (U.S. Pat. No. 4,489,160) or pNG2 (Serwold-Davis et al., FEMS Microbiology Letters 66: 119-124 (1990)) or pAG1 (U.S. Pat. No. 5,158,891), can be used in the same way.

Moreover, plasmid vectors which can be used for applying the method of gene amplification through integration in the chromosome, are also suitable, as was described for example by Reinscheid et al. (Applied and Environmental Microbiology 60: 126-132 (1994)) for duplication or amplification of the hom-thrB operon. In this method the complete gene is cloned into a plasmid vector, which can be replicated in a host (typically Escherichia coli), but not in Corynebacterium glutamicum. For example pSUP301 (Simon et al., Bio/Technology 1: 784-791 (1983)), pK18mob or pK19mob (Schäfer et al., Gene 145: 69-73 (1994)), pGEM-T (Promega Corporation, Madison, Wis., USA), pCR2.1—TOPO (Shuman, Journal of Biological Chemistry 269: 32678-84 (1994)), pCR® Blunt (Invitrogen, Groningen, The Netherlands), pEM1 (Schrumpf et al., Journal of Bacteriology 173: 4510-4516)) or pBGS8 (Spratt et al., Gene 41: 337-342 (1986)) may be considered as vectors. The plasmid vector that contains the gene to be amplified is then transferred by conjugation or transformation into the desired strain of Corynebacterium glutamicum. The method of conjugation is described for example in Schäfer et al., Applied and Environmental Microbiology 60: 756-759 (1994). Methods of transformation are described for example in Thierbach et al., Applied Microbiology and Biotechnology 29: 356-362 (1988), Dunican and Shivnan, Bio/Technology 7: 1067-1070 (1989) and Tauch et al., FEMS Microbiology Letters 123: 343-347 (1994). After homologous recombination by means of a “cross-over” event, the resultant strain contains at least two copies of the gene in question.

The formulation “an increased activity of an enzyme Eₓ relative to its wild type” used above and in the subsequent account preferably always means an activity of the particular enzyme Eₓ increased by a factor of at least 2, especially preferably of at least 10, more preferably of at least 100, even more preferably of at least 1000 and most preferably of at least 10 000. Furthermore, the cell according to the invention, which has “an increased activity of an enzyme Eₓ relative to its wild type”, in particular also comprises a cell whose wild type displays no or at least no detectable activity of this enzyme Eₓ and does not display a detectable activity of this enzyme Eₓ until after enzyme activity has increased, for example through overexpression. In this context the term “overexpression” or the formulation “increase in expression” used in the subsequent account also covers the case when a starting cell, for example a wild-type cell, has no or at least no detectable expression and a detectable expression of the enzyme Eₓ is only induced by recombinant techniques.

The formulation “reduced activity of an enzyme E_{X}” used below accordingly means preferably an activity that is reduced by a factor of at least 0.5, especially preferably of at least 0.1, more preferably of at least 0.01, even more preferably of at least 0.001 and most preferably of at least 0.0001. The reduction in activity of a particular enzyme can for example take place through directed mutation, by adding competitive or non-competitive inhibitors or through other measures known by a person skilled in the art for reducing the expression of a particular enzyme.

According to a first variant of the cell according to the invention, the latter has, in addition to the increased activity of the enzyme E₁, also an increased activity of at least one of the following enzymes E₂ to E₈:
of an enzyme E₂, which catalyses the conversion of two acetyl-coenzyme A units to acetoacetyl-coenzyme A;
of an enzyme E₃, which catalyses the conversion of acetoacetyl-coenzyme A to 3-hydroxybutyrate;
of an enzyme E₄, which catalyses the conversion of 3-hydroxybutyrate to crotonyl-coenzyme A;
of an enzyme E₅, which catalyses the conversion of ethylmalonyl-coenzyme A to methylsuccinyl-coenzyme A;
of an enzyme E₈, which catalyses the conversion of methylsuccinyl-coenzyme A to mesaconyl-coenzyme A;
of an enzyme E₇, which catalyses the conversion of mesaconyl-coenzyme A to mesaconyl-coenzyme A to β-methylmalyl-coenzyme A;
of an enzyme E₈, which catalyses the conversion of β-methylmalyl-coenzyme A to glyoxylate and propionyl-coenzyme A.

Cells that are especially preferred according to the invention are those in which, in addition to the activity of the enzyme E₁, the activity of the following enzymes or combinations of enzymes is increased: E₂, E₃, E₄, E₅, E₆, E₇, E₈, E₂E₃, E₂E₄, E₂E₅, E₂E₆, E₂E₇, E₂E₈, E₃E₄, E₃E₅, E₃E₆, E₃E₇, E₃E₈, E₄E₅, E₄E₆, E₄E₇, E₄E₈, E₅E₆, E₅E₇, E₅E₈, E₆E₇, E₆E₈, E₇E₈ and E₂E₃E₄E₅E₆E₇E₈. It is then basically possible and also preferable to use a cell whose wild type already displays one or optionally already all of the above enzyme activities, for example Rhodobacter sphaeroides, and in this wild type then to increase either only the activity of the enzyme E₁ or, in addition, one of the, several of the or all enzyme activities E₂ to E₈ using recombinant methods. Basically, however, it is also possible to use a cell whose wild type does not have any of the aforementioned enzyme activities E₁ to E₈, and in which all these activities are then increased by recombinant methods.

In this connection it is especially preferable if the enzyme

E₂ is a β-ketothiolase (EC 2.3.1.9),
E₃ is an acetoacetyl-coenzyme A-reductase (EC 1.1.1.36),
E₄ is an enoyl-coenzyme A-hydratase (EC 4.2.1.17),
E₅ is an ethylmalonyl-coenzyme A-mutase (EC 5.4.99.2),
E₆ is a methylsuccinyl-coenzyme A-dehydrogenase,
E₇ is a mesaconyl-coenzyme A-hydratase, and
E₈ is a β-methylmalyl/L-malyl-coenzyme A-lyase.

The enzyme E₂ is preferably encoded by genes selected from the group comprising acat1, acat2, loc484063, loc489-421, mgc69098, mgc81403, mgc81256, mgc83664, kat-1, erg10, ygeF, atoB, fadAx, phbA-1, phbA-2, atoB-2, pcaF, pcaF-2, phb-A, bktB, phaA, tioL, thlA, fadA, paaJ, phbAf, pimB, mmgA, yhfS, thl, vraB, thl, mvaC, thiL, paaJ, fadA3, fadA4, fadA5, fadA6, cg112392, catF, sc8f4.03, thiL1, thiL2, acaB1, acaB2, acaB3 or acaB4, with acat1, acat2, atoB and phbA and the corresponding gene from Rhodobacter sphaeroides being especially preferred.

The enzyme E₃ is preferably encoded by genes selected from the group comprising phbB, fabG, phbN1, phbB2 or cg112444, with phbB especially and the corresponding gene from Rhodobacter sphaeroides being especially preferred.

The enzyme E₄ is preferably encoded by genes selected from the group comprising echS1, ehhadh, hadha, echs1-prov, cg4389, cg4389, cg6543, cg6984, cg8778, ech-1, ech-2, ech-3, ech-4, ech-5, ech-6, ech-7, FCAALL.314, fcaall.21, fox2, eci1, eci2, paaF, paaG, yfcX, fadB, faoA, rpfF, phaA, phaB, echA1, echA2, echA3, echA4, echA5, echA6, echA7, echA8, echA9, echA9, echA10, echA11, echA12, echA13, echA14, echA15, echA16, echA17, echA18, echA19, echA20, echA21, fad-1, fad-2, fad-3, fad-4, fad-5, dcaE, hcaA, fadJ, rsp0671, rsp0035, rsp0648, rsp0647, rs03234, rs03271, rs04421, rs04419, rs02820, rs02946, paaG1, paaG2, paaG3, ech, pksH, ydbS, eccH1, ecCH2, pimF, fabJ1, fabJ2, caiD2, ysiB, yngF, yusL, fucA, cg10919, scf41.23, scd10.16, sck13.22, scp8.07c, stbac16h6.14, sc5f2a.15, sc6a5.38, hbd-1, hbd-2, hdb-3, hdb-4, hdb-5, hdb-6, hdb-7, hdb-8, hdb-9, hdb-10, paaF-1, paaF-2, paaF-3, paaF-4, paaF-5, paaF-6, paaF-7 and crt, with the corresponding gene from Rhodobacter sphaeroides being especially preferred.

Suitable genes for the enzyme E₅ are selected from the group comprising mut, mutA, mutB, sbm, sbmA, sbmB, sbm5, bhbA, mcmA, mcmA1, mcmA2, mcmB, mcm1, mcm2, mcm3, icmA, meaA1 and meaA2, with once again the corresponding gene from Rhodobacter sphaeroides being especially preferred.

Preferred genes for the enzymes E₆, E₇ and E₈ are in particular the genes for these enzymes from Rhodobacter sphaeroides.

Examples of nucleotide sequences of the aforementioned genes and other genes for the enzymes E₂ to E₈ can also be taken inter alia from the KEGG database, the NCBI database or EMBL database.

According to a first particular embodiment of the first variant of the cell according to the invention, in which the activity of the enzyme E₁ and optionally also the activity of one of the, several of the or all of the enzymes E₂ to E₈ is increased, this additionally displays an increased activity of one or more of the following enzymes E₉ to E₁₂:
of an enzyme E₉, which catalyses the conversion of propionyl-coenzyme A to methylmalonyl-coenzyme A;
of an enzyme E₁₀, which catalyses the conversion of methylmalonyl-coenzyme A to methylmalonate;
of an enzyme Eₙ, which catalyses the conversion of methylmalonate to methylmalonate-semialdehyde;
of an enzyme E₁₂, which catalyses the conversion of methylmalonate-semialdehyde to 3-hydroxyisobutyric acid.

Cells that are especially preferred according to the invention are those for which, in addition to the activity of the enzyme E₁ and of one or more of the activities E₂ to E₈, the activity of the following enzymes or combinations of enzymes is increased: E₉, E₁₀, E₁₁, E₁₂, E₉E₁₀, E₉E₁₁, E₉E₁₂, E₁₀E₁₁, E₁₀E₁₂, E₁₁E₁₂, E₉E₁₀E₁₁, E₉E₁₀E₁₂, E₉E₁₁E₁₂, E₁₀E₁₁E₁₂ and E₉E₁₀E₁₁E₁₂, with the combination E₉E₁₀E₁₁E₁₂ being the most preferred.

In this context it is especially preferable if the enzyme
E₉ is a propionyl-coenzyme A-carboxylase (EC 6.4.1.3),
E₁₀ is a methylmalonyl-coenzyme A-hydrolase (EC 3.1.2.17),
E₁₁ is an aldehyde-dehydrogenase (EC 1.2.1.3) or an aldehyde-oxidase (EC 1.2.3.1) and
E₁₂ is a 3-hydroxyisobutyrate-dehydrogenase (EC 1.1.1.31) or a 3-hydroxyacyl-coenzyme A-dehydrogenase (EC 1.1.1.35).

Basically it is conceivable to use four mutually independent enzymes E₉ to E₁₂, or to use an enzyme complex, with which at least two of the above reactions brought about by the enzymes E₉ to E₁₂ can be carried out. In particular, enzyme complexes should be mentioned which catalyse both the conversion of methylmalonyl-coenzyme A to methylmalonate as well as the subsequent conversion of methylmalonate to methylmalonate-semialdehyde.

Suitable genes for the enzyme E₉ are selected from the group comprising pccA, pccB, accD1, accD, rs03236, accB, accC, pycA, ygjD, yngE, pcc, accA2, accD1, accD2, accD3, accD4, accD5, accD6, bccA1, pccB1, pccB4, pccB5, cg110707, cg110708, cg112870, dtsR, dtsR1, dtsR2, scd10.12, scd10.13, mccB and mmdA, with pccA and pccB being especially preferred.

The enzyme E₁₀ is preferably encoded by the aox1 gene. Methylmalonyl-coenzyme A-hydrolase from rat liver is described for example in Kovachy et al., “Recognition, isolation, and characterization of rat liver D-methylmalonyl coenzyme A hydrolase”, J. Biol. Chem. 258 (1983), pages 11415-11421.

The enzyme E₁₁ is preferably encoded by genes selected from the group comprising acat1, acat2, loc484063, loc489-421, mgc69098, mgc81403, mgc81256, mgc83664, kat-1, erg10, ygeF, atoB, fadAx, phbA-1, phbA-2, atoB-2, pcaF, pcaF-2, phb-A, bktB, phaA, tioL, thlA, fadA, paaJ, phbAf, pimB, mmgA, yhfS, thl, vraB, thl, mvaC, thiL, fadA3, fadA4, fadA5, fadA6, cg112392, catF, sc8f4.03, thiL1, thiL2, acaB1, acaB2, acaB3, acaB4 or, with acat1, acat2 and atoB being especially preferred.

Suitable genes for the enzyme E₁₂ are selected from the group comprising hibadh, cg15093, cg15093, cg4747, mwL2.23, t13k14.90, f19b15.150, hibA, ygbJ, mmsB, garR, tsar, mmsB-1, mmsB-2, yfjR, ykwC, ywjF, hibD, glxR, SCM1.40c, ehhand, hadh2, hadhsc, hsd17B4, loc488110, had, mgC81885, hadh2-prov, cg3415, cg7113, ech-1, ech-8, ech-9, ard-1, yfcX, fadB, faoA, fadB2x, hbd-1, hbd-2, hbd-3, hbd-4, hbd-5, hbd-6, hbd-7, hbd-8, hbd-9, hbd-10, fadJ, rs04421, rs02946, rs05766, bbsD, bbsC, fadB1, fadB2, fadB5, hbdA, pimF, fabJ-1, fabJ, scbac19f3.11, sci35.13, scbac8d1.10c, sc5f2a.15, sc6a5.38, fadC2, fadC4, fadC5, fadC6, had and paaH. Other suitable 3-hydroxyisobutyrate-dehydrogenases are described for example in Bannerjee et al. (1970), J. Biol. Chem., 245, pages 1828 to 1835, Steele et al. (1992), J. Biol. Chem., 267, pages 13585 to 13592, Harris et al. (1988), J. Biol. Chem., 263, pages 327 to 331, Harris et al., Biochim. Biophys. Acta, 1645 (1), pages 89 to 95, Hawes et al. (2000), Methods Enzymol., 324, pages 218 to 228, Harris et al., J. Biol. Chem., 275 (49), pages 38780 to 38786, Rougraff et al. (1988), J. Biol. Chem., 263(1), pages 327 to 331, Robinson et al., J. Biol. Chem., 225, pages 511 to 521, Hawes et al. (1995), Biochemistry, 34, pages 4231 to 4237, Hasegawa J. (1981), Agric. Biol. Chem., 45, pages 2805 to 2814, Hawes et al. (1996), FEBS Lett., 389, pages 263 to 267, Hawes et al. (1996), Enzymology and Molecular Biology of Carbonyl Metabolism, Plenum Press, New York, pages 395 to 402, Adams et al. (1994), Structure, 2, pages 651 to 668, Zhang et al. (1999), Biochemistry, 38, pages 11231 to 11238, Mirny et al., (1999), J. Mol. Biol., 291, pages 177 to 196 and Lokanath et al. (2005), J Mol. Biol. The disclosure of these publications is introduced hereby as reference and forms part of the disclosure of the present invention.

According to an especially preferred embodiment of the first variant of the cell according to the invention, in which one or more of the enzyme activities E₉ to E₁₂ is increased, the enzyme E₁₁ is encoded by a DNA sequence that is selected from the following sequences:
A) from a sequence according to SEQ ID No. 03,
B) from an intron-free sequence, which is derived from a sequence according to A) and which encodes the same protein or peptide as the sequence according to SEQ ID No. 03,
C) from a sequence that encodes a protein or peptide which comprises the amino acid sequence according to SEQ ID No. 04,
D) from a sequence that is at least 80%, especially preferably at least 90%, more preferably at least 95% and most preferably at least 99% identical to a sequence according to one of the groups A) to C), especially preferably according to group a), and this sequence preferably encodes a protein or peptide that is capable of converting both methylmalonate and malonate to the corresponding semialdehydes, methylmalonate-semialdehyde or malonate-semialdehyde,
E) from a sequence that hybridizes with the antisense strand of a sequence according to one of the groups A) to D), especially preferably according to group A), or would hybridize taking into account degeneration of the genetic code, and this sequence preferably encodes a protein or peptide, that is capable of converting both methylmalonate and malonate to the corresponding semialdehydes, methylmalonate-semialdehyde or malonate-semialdehyde,
F) from a derivative of a sequence according to one of the groups A) to D), especially preferably according to group A), obtained by substitution, addition, inversion and/or deletion of at least one base, preferably of at least 2 bases, more preferably of at least 5 bases and most preferably at least 10 bases, though preferably of not more than 100 bases, especially preferably of not more than 50 bases and most preferably of not more than 25 bases, and this derivative preferably encodes a protein or peptide that is capable of converting both methylmalonate and malonate to the corresponding semialdehydes, methylmalonate-semialdehyde or malonate-semialdehyde,
G) from a sequence that corresponds to SEQ ID No. 03 within the degeneration of the genetic code, and
H) from a sequence with neutral sense mutations of SEQ ID No. 03.

The nucleic acid sequence described above is the gene for a methylmalonyl-coenzyme A-reductase or malonyl-coenzyme A-reductase from Sulfolobus tokodaii, which is able to convert methylmalonate or malonate to the corresponding semialdehydes especially efficiently. This enzyme puts into effect both the activity of the enzyme E₁₀ and that of the enzyme E₁₁.

According to an especially preferred embodiment of the first variant of the cell according to the invention, this accordingly has, in comparison with its wild type, at least one increased activity of the enzymes E₁ and E₁₁, with E₁ being encoded by a DNA sequence according to one of the alternatives a) to h) and the enzyme E₁₁ by a DNA sequence according to one of the alternatives A) to H). In this connection it is preferable if the increased activity of these two enzymes is achieved in that the polypeptides with SEQ ID No. 02 and SEQ ID No. 04 or alternatively in that amino acid sequences having identity of at least 50%, preferably at least 55%, more preferably at least 60%, even more preferably at least 65% and most preferably at least 70% to the amino acid sequence according to SEQ ID No. 02 or SEQ ID No. 04, are overexpressed in the cell. These two DNA sequences can then be integrated in the genome of the cell or can be present on a vector inside the cell.

According to a second particular embodiment of the first variant of the cell according to the invention, in which the activity of the enzyme E₁ and optionally also the activity of one of the, several of the or all of the enzymes E₂ to E₈ are increased, this additionally displays an increased activity of one or more of the following enzymes E₁₃ and E₁₂:
of an enzyme Eₙ, which catalyses the conversion of propionyl-coenzyme A to methylmalonate-semialdehyde;
of an enzyme E₁₂, which catalyses the conversion of methylmalonate-semialdehyde to 3-hydroxyisobutyric acid.

Cells that are especially preferred according to the invention are then those for which, in addition to the activity of the enzyme E₁ and of one or more of the activities E₂ to E₈, the activity of the following enzymes or combinations of enzymes is increased: E₁₂, E₁₃ and E₁₂E₁₃, with the combination E₁₂E₁₃ being most preferred.

In this connection it is especially preferable if the enzyme
E₁₃ is a methylmalonate-semialdehyde-dehydrogenase (EC 1.2.1.27), and
E₁₂ is a 3-hydroxyisobutyrate-dehydrogenase (EC 1.1.1.31) or a 3-hydroxyacyl-coenzyme A-dehydrogenase (EC 1.1.1.35).

Suitable genes for the enzyme E₁₃ are preferably selected from the group comprising aldh6a1, cg17896, t22c12.10, ald6, putA1, mmsA, mmsA-1, mmsA-2, mmsA-3, mmsA-4, msdA, iolA and iolAB, with mmsA being especially preferred.

Suitable genes for the enzyme E₁₂ are preferably those that have already been mentioned in connection with the first particular embodiment of the first variant of the cell according to the invention.

The nucleotide sequences of the aforementioned genes for the enzymes E₁₂ and E₁₃ can inter alia also be taken from the KEGG database.

It is further preferred, in connection with the first variant of the cell according to the invention, to use such cells as are especially capable of utilizing C₁-carbon sources via the serine cycle. Once again, the methylotrophic and methanotrophic microorganisms already mentioned at the beginning are especially preferred.

According to a second variant of the cell according to the invention, in addition to the increased activity of the enzyme E₁, this also has an increased activity of at least one of the following enzymes E₁₄, E₁₅ and E₁₀ to E₁₂:
of an enzyme E₁₄, which catalyses the conversion of beta-alanine to beta-alanyl-coenzyme A,
of an enzyme E₁₅, which catalyses the conversion of beta-alanyl-coenzyme A to acrylyl-coenzyme A,
of an enzyme E₁₀, which catalyses the conversion of methylmalonyl-coenzyme A to methylmalonate;
of an enzyme E₁₁, which catalyses the conversion of methylmalonate to methylmalonate-semialdehyde;
of an enzyme E₁₂, which catalyses the conversion of methylmalonate-semialdehyde to 3-hydroxyisobutyric acid.

Cells that are especially preferred according to the invention are those for which, in addition to the activity of the enzyme E₁, the activity of the following enzymes or combinations of enzymes is increased: E₁₄, E₁₅, E₁₀, E₁₁, E₁₂, E₁₄E₁₅, E₁₄E₁₀, E₁₄E₁₁, E₁₄E₁₂, E₁₅E₁₀, E₁₅E₁₁, E₁₅E₁₂, E₁₀E₁₁, E₁₀E₁₂, E₁₁E₁₂ and E₁₄E₁₅E₁₀E₁₁E₁₂. It is moreover basically possible to use a cell that is already capable of forming especially large amounts of acrylyl-coenzyme A.

In this connection it is especially preferable if the enzyme
E₁₄ is a coenzyme A-transferase (EC 2.8.3.1) or coenzyme A-synthetase, preferably a coenzyme A-transferase,
E₁₅ is a beta-alanyl-coenzyme A-ammonium-lyase (EC 4.3.1.6),
E₁₀ is a methylmalonyl-coenzyme A-hydrolase (EC 3.1.2.17),
E₁₁ is an aldehyde-dehydrogenase (EC 1.2.1.3) or an aldehyde-oxidase (EC 1.2.3.1) and
E₁₂ is a 3-hydroxyisobutyrate-dehydrogenase (EC 1.1.1.31) or a 3-hydroxyacyl-coenzyme A-dehydrogenase (EC 1.1.1.35).

Preferred enzymes E₁₄ with CoA-transferase activity are those from Megasphaera elsdenii, Clostridium propionicum, Clostridium kluyveri and also from Escherichia coli. As examples of a DNA sequence encoding a CoA-transferase we may mention at this point the sequence from Megasphaera elsdenii designated with SEQ ID No. 24 in WO-A-03/062173. Other preferred enzymes are those variants of CoA-transferase that are described in WO-A-03/062173.

Suitable enzymes E₁₅ with a beta-alanyl-coenzyme A-ammonium-lyase activity are for example those from Clostridium propionicum. DNA sequences that encode such an enzyme can for example be obtained from Clostridium propionicum, as described in Example 10 in WO-A-03/062173. The DNA sequence that encodes the beta-alanyl-coenzyme A-ammonium-lyase from Clostridium propionicum is given in WO-A-03/062173 as SEQ ID No. 22.

Suitable genes for the enzymes E₁₀ to E₁₂ have already been mentioned in connection with the first variant of the cell according to the invention, and in connection with the second variant it is also preferable, as gene for the enzyme Eₙ, the gene described above from Sulfolobus tokodaii is especially preferred.

In connection with the second variant of the method according to the invention it may moreover be advantageous if, in addition to the increase in activity of the enzyme E₁, the activity of one or more of the enzymes E₂ to E₈ and the activity of one or more of the enzymes E₁₄, E₁₅ and E₉ to E₁₂, the cell has at least one, and preferably both of the following properties:
an increased activity, in comparison with its wild type, of an enzyme E₁₆ₐ, which catalyses the conversion of pyruvate to oxaloacetate or of an enzyme E_{16b}, which catalyses the conversion of phosphoenolpyruvate to oxaloacetate, though preferably of an enzyme E₁₆ₐ, which catalyses the conversion of pyruvate to oxaloacetate, and
an increased activity of an enzyme E₁₋₇, which catalyses the conversion of aspartate to beta-alanine,

The enzyme E₁₆ₐ is preferably a carboxylase, especially preferably a pyruvate carboxylase (EC-number 6.4.1.1), which catalyses the conversion of pyruvate to oxaloacetate. A pyruvate carboxylase that is especially preferred in this connection is the mutant that is described in “A novel methodology employing Corynebacterium glutamicum genome information to generate a new L-lysine-producing mutant.”, Ohnishi J et al., Applied Microbiology and Biotechnology, Vol. 58 (2), pages 217-223 (2002). In this mutation the amino acid proline in position 458 was replaced by serine. The disclosure of this publication concerning the possibilities for the production of pyruvate-carboxylase mutants is hereby introduced as reference and forms part of the disclosure of the present invention.

The enzyme E₁₇ is preferably a decarboxylase, especially preferably a glutamate decarboxylase or an aspartate decarboxylase, with an 1-aspartate-1-decarboxylase (EC-number 4.1.1.11) being most preferred, which is encoded by the panD gene. Aspartate decarboxylase catalyses the conversion of aspartate to beta-alanine. Genes for aspartate decarboxylase (panD genes) have already been cloned and sequenced from, among others, Escherichia coli (FEMS Microbiology Letters, 143, pages 247-252 (1996)), “Photorhabdus luminescens subsp. Laumondii, Mycobacterium bovis subsp. Bovis”) and from numerous other microorganisms. In particular the nucleotide sequence of the panD gene from Corynebacterium glutamicum is described in DE-A-198 55 313. Basically it is possible to use panD genes from any conceivable origin, whether from bacteria, yeasts or fungi. Furthermore, all alleles of the panD gene can be used, in particular also those that result from the degeneracy of the genetic code or from function-neutral sense mutations. Apart from the aspartate decarboxylase from Corynebacterium glutamicum, an aspartate decarboxylase especially preferred according to the invention is the Escherichia coli mutant DV9 (Vallari and Rock, Journal of Bacteriology, 164, pages 136-142 (1985)). The disclosure of this publication regarding the aforementioned mutant is hereby introduced as reference and forms part of the disclosure of the present invention.

A further contribution to solution of the problems mentioned at the beginning is provided by a method of production of a genetically engineered cell, comprising the step of increasing the activity of the enzyme E₁, which is encoded by a DNA sequence according to one of the groups a) to h), as defined at the beginning, or which possesses the amino acid sequence with SEQ ID No. 02 or an amino acid sequence that is at least 50%, preferably at least 55%, more preferably at least 60%, even more preferably at least 65% and most preferably at least 70% identical to the amino acid sequence according to SEQ ID No. 02, in a cell. Preferably the activity of the enzyme E₁ is increased by inserting the DNA sequence according to one of the groups a) to h), preferably a to f) as an exogenous DNA sequence into a cell and then initiating the expression of the polypeptide that is encoded by this DNA sequence.

Optionally the method further comprises increasing the activity of one or more of the activities E₂ to E₁₇, in particular also increasing the enzyme Eₙ, which is encoded by a DNA sequence according to one of the groups A) to H), as defined at the beginning, or which has the amino acid sequence with SEQ ID No. 04 or an amino acid sequence that is at least 50%, preferably at least 55%, more preferably at least 60%, even more preferably at least 65% and most preferably at least 70% identical to the amino acid sequence according to SEQ ID No. 04, in a cell.

A contribution to solution of the problems mentioned at the beginning is also provided by a genetically engineered cell, which can be obtained by the method described above.

A contribution to solution of the problems mentioned at the beginning is further provided by the use of the cell according to the invention, described above, for the production of ethylmalonyl-coenzyme A or methylmalonyl-coenzyme A, preferably as intermediates for the production of 3-hydroxyisobutyric acid, or alternatively directly for the production of 3-hydroxyisobutyric acid or derivatives thereof.

A contribution to solution of the problems mentioned at the beginning is, moreover, provided by a method of production of 3-hydroxyisobutyric acid or derivatives thereof, comprising the steps:
contacting the cell according to the invention, described above, with a culture medium containing a source of carbon, for example carbohydrates, glycerol, carbon dioxide, methane, methanol, lipids, L-valine or L-glutamate, in conditions in which 3-hydroxyisobutyric acid is formed from the carbon source;
purification of the 3-hydroxyisobutyric acid or derivative thereof thus obtained.

The genetically engineered cells according to the invention can be brought in contact with the nutrient medium and thus cultivated continuously or discontinuously in a batch process or in a fed-batch process or a repeated-fed-batch process for the purpose of producing the aforementioned products. A semi-continuous process, as described in GB-A-1009370, is also conceivable. Known cultivation methods are summarized in Chmiel's textbook (“Bioprozesstechnik 1. Einfuhrung in die Bioverfahrenstechnik” (Gustav Fischer Verlag, Stuttgart, 1991)) or in Storhas' textbook (“Bioreaktoren and periphere Einrichtungen”, Vieweg Verlag, Braunschweig/Wiesbaden, 1994).

The culture medium to be used must suitably satisfy the requirements of the particular strains. Descriptions of culture media of various microorganisms are given in “Manual of Methods for General Bacteriology” of the American Society for Bacteriology (Washington D.C., USA, 1981).

Carbohydrates such as glucose, sucrose, lactose, fructose, maltose, molasses, starch and cellulose, oils and fats such as soya oil, sunflower oil, peanut oil and coconut oil, fatty acids such as palmitic acid, stearic acid and linolic acid, alcohols such as glycerol and methanol, hydrocarbons such as methane, amino acids such as L-glutamate or L-valine or organic acids such as acetic acid, can be used as the carbon source. These substances can be used separately or as a mixture. The use of carbohydrates, in particular monosaccharides, oligosaccharides or polysaccharides, as described in U.S. Pat. No. 6,01,494 and U.S. Pat. No. 6,136,576, of C₅ sugars or of glycerol, is especially preferred.

Especially when the cells are cells that are capable of utilizing C₁-carbon sources via the serine cycle, it is preferable to add carbon sources such as methanol or methane to the culture medium.

Organic nitrogen-containing compounds such as peptones, yeast extract, meat extract, malt extract, corn-steep liquor, soybean flour and urea or inorganic compounds such as ammonium sulphate, ammonium chloride, ammonium phosphate, ammonium carbonate and ammonium nitrate can be used as the nitrogen source. The nitrogen sources can be used separately or as a mixture.

Phosphoric acid, potassium dihydrogenphosphate or dipotassium hydrogenphosphate or the corresponding sodium-containing salts can be used as the source of phosphorus. The culture medium must in addition contain metal salts such as magnesium sulphate or iron sulphate, which are required for growth. Finally, essential growth substances such as amino acids and vitamins can be used in addition to the aforementioned substances. In addition, suitable precursors can be added to the culture medium. The stated materials can be added to the culture as a single addition, or can be supplied in a suitable manner during cultivation.

For control of the culture pH, basic compounds such as sodium hydroxide, potassium hydroxide, ammonia or ammonia water or acid compounds such as phosphoric acid or sulphuric acid can be used in a suitable manner. Antifoaming agents such as fatty acid polyglycol esters can be used for controlling foaming. Suitable selectively acting substances such as antibiotics can be added to the medium to maintain the stability of plasmids. In order to maintain aerobic conditions, oxygen or oxygen-containing gas mixtures such as air are fed into the culture.

The temperature of the culture is normally above 20° C., preferably above 30° C., it can even be above 40° C., and preferably a cultivation temperature of 95° C., especially preferably 90° C. and most preferably 80° C. is not exceeded.

This purification of 3-hydroxyisobutyric acid can be carried out by any purification technique known by a person skilled in the art. For example sedimentation, filtration or centrifugation techniques can be used, for first separating the cells from the culture medium. The 3-hydroxyisobutyric acid can be isolated by extraction, distillation or ion-exchange from the cell-free culture medium containing 3-hydroxyisobutyric acid.

The purification of 3-hydroxyisobutyric acid from the nutrient solution is, according to a particular embodiment of the method according to the invention, carried out continuously, and in this connection it is moreover preferable for the fermentation to be carried out continuously as well, so that the entire process from the enzymatic reaction of the educts with formation of 3-hydroxyisobutyric acid to the purification of the 3-hydroxyisobutyric acid from the culture medium can be carried out continuously. For continuous purification of the 3-hydroxyisobutyric acid from the culture medium, this is conducted continuously using a device for removing the cells employed during fermentation, preferably using a filter with an exclusion size in a range from 20 to 200 kDa, in which solid/liquid separation takes place. It is also conceivable to use a centrifuge, a suitable sedimentation device or a combination of these devices, and it is especially preferable to remove at least some of the cells first by sedimentation and then feed the culture medium, from which the cells have partially been removed, to an ultrafiltration or centrifugation device.

The fermentation product, which now has a higher proportion of 3-hydroxyisobutyric acid, is fed—after removal of the cells—to a preferably multistage separator. This separator has several successive separation stages, from which return lines provide recycling to the second fermentation tank. In addition there are discharge lines from the individual separation stages. The individual separation stages can operate according to the principle of electrodialysis, reverse osmosis, ultrafiltration or nanofiltration. As a rule the individual separation stages comprise membrane separators. The individual separation stages are selected based on the type and amount of fermentation by-products and substrate residues.

The invention will now be explained in more detail on the basis of non-limiting diagrams and examples.

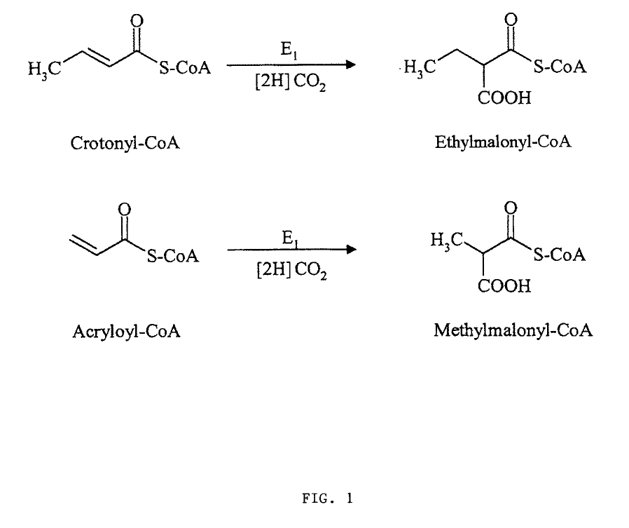

FIG. 1 shows the reactions catalysed by crotonyl-coenzyme A reductase/carboxylase from Rhodobacter sphaeroides.

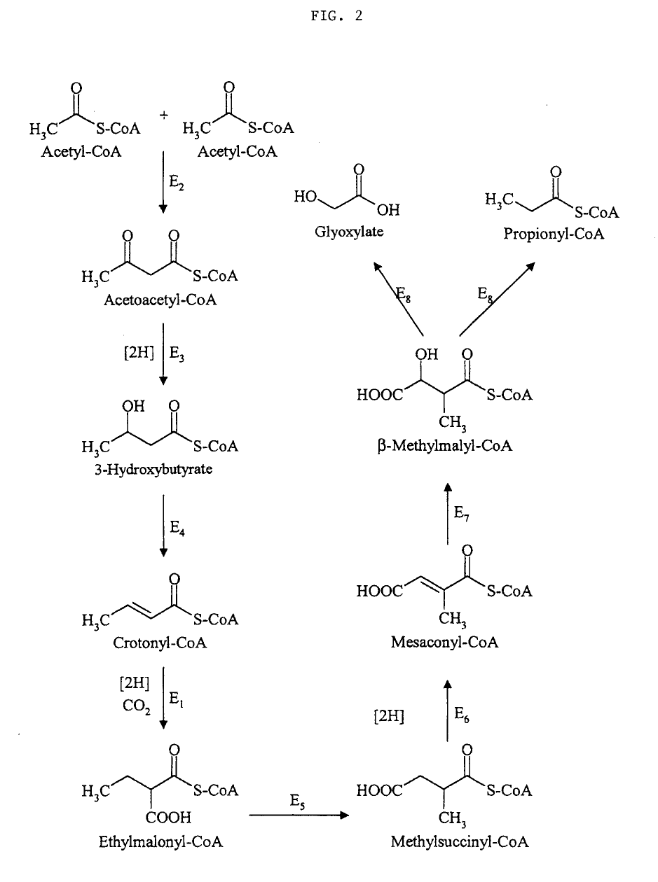

FIG. 2 shows the ethylmalonyl-coenzyme A metabolic pathway in Rhodobacter sphaeroides.

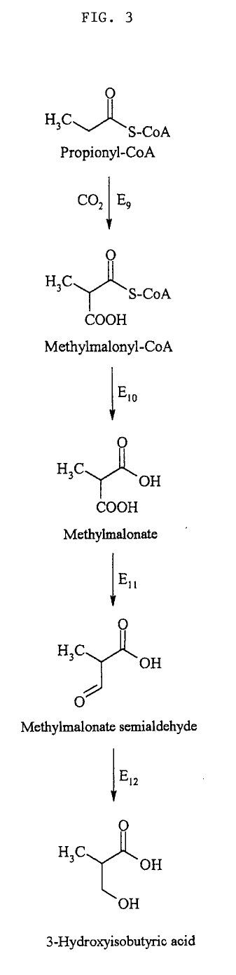

FIG. 3 shows a particular embodiment of the first variant of the cell according to the invention, in which crotonyl-coenzyme A reductase/carboxylase catalyses the conversion of crotonyl-coenzyme A to ethylmalonyl-coenzyme A and in which the propionyl-coenzyme A subsequently formed is converted via methylmalonate and methylmalonate-semialdehyde to 3-hydroxyisobutyric acid.

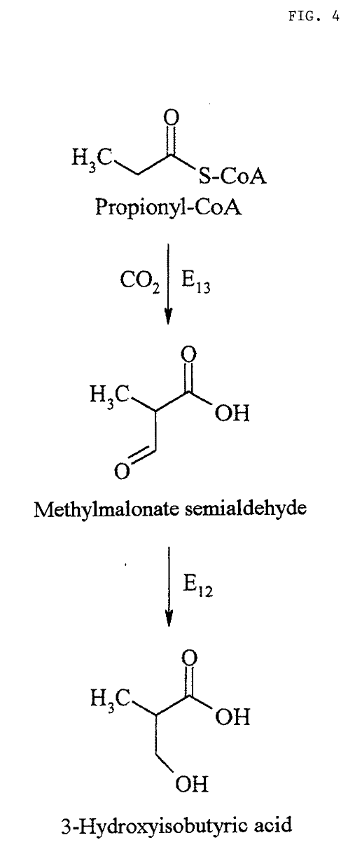

FIG. 4 shows another particular embodiment of the first variant of the cell according to the invention, in which crotonyl-coenzyme A reductase/carboxylase catalyses the conversion of crotonyl-coenzyme A to ethylmalonyl-coenzyme A and in which the propionyl-coenzyme A subsequently formed is converted via methylmalonate-semialdehyde to 3-hydroxyisobutyric acid.

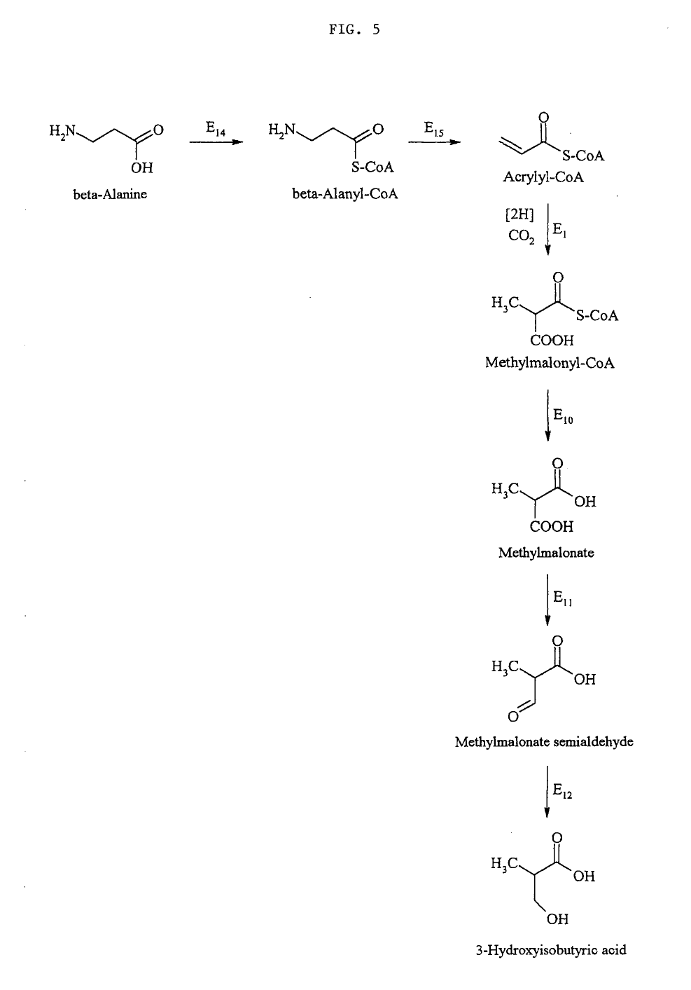

FIG. 5 shows a particular embodiment of the second variant of the cell according to the invention, in which crotonyl-coenzyme A reductase/carboxylase catalyses the conversion of acrylyl-coenzyme A to methylmalonyl-coenzyme A.

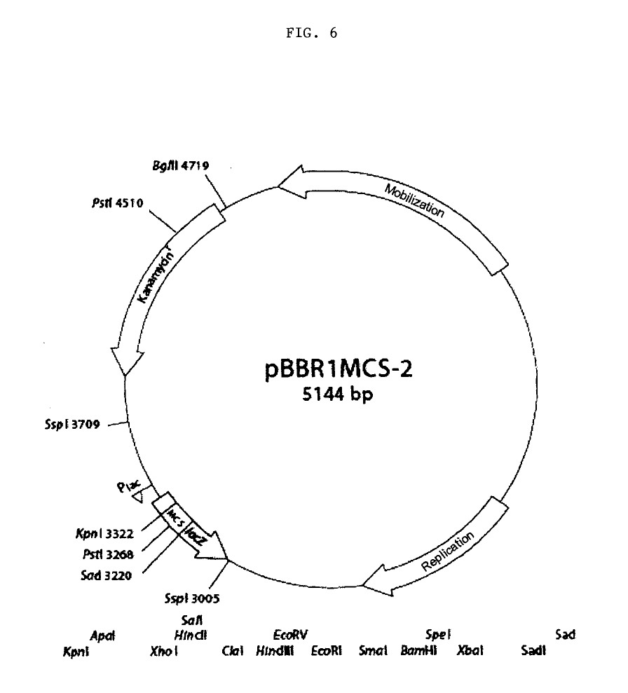

FIG. 6 shows the vector map of the expression plasmid used in Example 2.

### EXAMPLES

### 1. Isolation of Genomic DNA from R. sphaeroides

For isolation of the DNA according to the invention, first the chromosomal DNA was isolated from Rhodobacter sphaeroides according to F. M. Ausubel et al., “Current Protocols in Molecular Biology”, John Wiley and Sons, New York, 1987. In this DNA, a homologous nucleic acid sequence was identified, which encodes a protein that is 78% identical to the crotonyl-coenzyme A-reductase gene (ccr gene) from Methylobacterium extorquens, 41% identical to the ccr gene from S. collinus and 39% identical to the ccr gene from S. coelicolor. Using the synthetic polynucleotides 5′-GGAGGCAACCATGGCCCTCGA-CGTGCAGAG-3′ (forward primer; NcoI cleavage site at the start codon is underlined) and 5′-GAGACTTGCGGATCCCTC-CGATCAGGC-CTTGC-3′ (reverse primer; BamHI cleavage site after a stop codon is underlined) and the DNA isolated from Rhodobacter sphaeroides as template, the ccr gene was amplified by PCR (Mullis et al., Cold Spring Harbor Symp. Quant. Biol., 51, pages 263-273, 1986). Preparative PCR was used, employing Pfu-polymerase (Pfunds, Genaxxon). The Pfu-polymerase contains a 3′-5′ exonuclease (“proofreading”) function. 32 cycles, each with 45 seconds at 95° C., 30 seconds at 55° C. and 3 minutes at 72° C., were carried out. The PCR was carried out in a thermocycler (Biometra, Gottingen).

### 2. Production of an Expression Vector

The PCR product obtained in Example 1 was isolated and cloned into a pBBR1MCS-2 expression vector, as described in Kovach et al., “Four new derivatives of the broad-host-range cloning vector pBBR1MCS carrying different antibiotic-resistance cassettes”, Gene, 166, pages 175-176 (1995). For this, the DNA-fragment obtained after purification in Example 1 and the expression vector pBBR1MCS-2 were submitted to restriction with the enzymes XholI and HindIII and then ligated. Then competent Rhodobacter sphaeroides cells were transformed with the expression vector.

### 3. Expression and Purification of the CCR

For heterologous expression in E. coli and purification of the CCR, the gene was cloned into the expression vector pET3d, preserving the plasmid pTE13: the R. sphaeroides ccr gene was amplified by PCR using the oligonucleotide ccr-fw (5′-GGAGGCAACCATGGCCCTCGACGTGCAGAG-3′; NcoI recognition sequence underlined) and ccr-rev (5′-GAGACTTGCGGATCCCTCCGATCAGGCCTTGC-3′; BamHI recognition sequence underlined), using chromosomal DNA of the strain R. sphaeroides 2.4.1 (DSMZ 158) as template. The PCR product was ligated as NcoI/BamHI fragment into the vector pET3d cut with NcoI/BamHI (Merck, Germany), obtaining the plasmid pTE13. Competent E. coli BL21 (DE3) cells were transformed with pTE13 and cultivated at 37° C. in LB medium with 100 μg/ml Ampicill in a 200-litre fermenter (80 l/min air stream; stirrer speed 300 rpm). At OD₅₇₈=0.75 it was induced with 0.5 mM isopropylthiogalactopyranoside (IPTG). The cells were cultivated for 3.5 h, then harvested and stored in liquid nitrogen awaiting further processing.

Purification of the enzyme was carried out at 4° C. in two steps by DEAE chromatography and affinity chromatography. 9 g of frozen E. coli cells were resuspended in twice the volume of buffer A (20 mM TriS-HCl, pH 7.9) supplemented with 0.1 mg/l DNase I. The suspension was macerated by two passages through a French press at 137 MPa and was then centrifuged for 1 h at 100 000×g. 15 ml of supernatant (1.6 g total protein) was fed at a flow rate of 2.5 ml/min to a 30 ml DEAE-Sepharose Fast Flow Column (Amersham Biosciences) (equilibrated beforehand with 60 ml buffer A). The column was then washed with 90 ml buffer A, then with 135 ml buffer A with 50 mM KCl. Activity was eluted with 100 mM KCl in buffer A in a total volume of 195 ml. Active fractions were pooled, desalted, and concentrated on an Amicon YM 10 membrane (Millipore, Bedford, Mass.) by ultrafiltration to a volume of 20 ml. 1.5 ml of the concentrate thus obtained (17 mg total protein) was fed at a flow rate of 0.5 ml/min to a 10 ml Cibacron blue 3GA agarose 3000 CL column (Sigma-Aldrich), which had been equilibrated beforehand with 20 ml buffer A. The column was washed twice with 22 ml buffer A, followed by 37 ml buffer A with 100 mM

KCl and 37 ml buffer A with 200 mM KCl. Activity was eluted with 500 mM KCl in buffer A in a total volume of 30 ml. Active fractions were pooled, desalted, and concentrated on an Amicon YM 10 membrane (Millipore, Bedford, Mass.) by ultrafiltration to a volume of 1.5 ml. The protein (7.5 mg) was stored in 50% glycerol at −20° C.

### 4. Detection of the Activity of the CCR

The activity of the CCR was determined spectrophotometrically, by monitoring the crotonyl-CoA dependent oxidation of NADPH at 360 nm. (ε_{NADPH}=3400 M⁻¹ cm⁻¹). A cuvette with a layer thickness of 0.1 cm was used. The reaction mix (0.2 ml) contained 100 mM Tris-HCl (pH 7.9), 4 mM NADPH, 2 mM crotonyl-CoA, and 1-5 μg purified CCR. The reaction was started by adding 33 mM KHCO₃ or NaHCO₃. The specific activity determined for the conversion of crotonyl-CoA of the enzyme obtained in Example 3 was 103 U mg⁻¹ (crotonyl-CoA).

The Kₘ values for crotonyl-CoA and NaHCO₃ were determined by varying the concentration of NaHCO₃ (0.4-66.6 mM) or crotonyl-CoA (0.125-2.0 mM). The Kₘ value for NADPH was determined by incorporating [¹⁴C] bicarbonate in (acid-resistant) ethylmalonyl-CoA. The reaction mix (0.33 ml) contained 100 mM Tris-HCl (pH 7.9), 3 mM crotonyl-CoA, 3 mM NaHCO₃, 64 kBq ml⁻¹ NaH¹⁴CO₃, and 7 μg purified CCR. The reaction was started by adding NADPH (0.125-5 mM). It was stopped at various points of time, by transferring 50 μl of the reaction mix into 50 μl 1.5 M HClO₄. The samples were shaken overnight, to remove ¹⁴CO₂ that had not been incorporated, and the amount of ¹⁴C incorporated was determined by scintillation measurement. The following Kₘ values were found: crotonyl-CoA (0.4 mM); NADPH (0.7 mM); HCO₃⁻ (14 mM, pH 7.9); ethylmalonyl-CoA (0.2 mM). In addition, the activity of the CCR was determined with acrylyl-CoA instead of crotonyl-CoA as substrate. The CCR catalyses the following reaction: Acrylyl-CoA+NADPH+CO₂→(2S)-methylmalonyl-CoA⁻+NADP⁺. The test mix (0.12 ml) contained 80 mM Tris.HCl (pH 7.8), 30 mM NaHCO₃, 4.3 mM NADPH and 5-10 μg recombinant CCR. The reaction was started by adding 1.3 mM acrylyl-CoA. The change in absorption at 360 nm and 30° C. was measured in a 0.1 cm thick cuvette.

For the conversion of acrylyl-CoA, a specific activity of about 45 U mg⁻¹ was determined.

The apparent Kₘ value for acrylyl-CoA was measured in a radioactive test, in which the incorporation of ¹⁴CO₂ in acrylyl-CoA was quantified. The amount of acrylyl-CoA was varied and the relative rate of incorporation was determined by scintillation.

The test mix (0.12 ml) contained 80 mM Tris.HCl (pH 7.8), 30 mM NaHCO₃, 100 kBq H¹⁴CO₃, 5.2 mM NADPH and 5-10 μg recombinant ccr. The reaction was started by adding 0.07-2.2 mM acrylyl-CoA. After various points of time at 30° C. (10-120 s), 25 μl samples were taken from the reaction mixture, 500 μl 5% TCA was added and after 12 h of “shaking-out” (to remove the unfixated ¹⁴CO₂) measurement was carried out in the scintillation counter. A Kₘ (acrylyl-CoA): 0.47 mM was found.

### 5. Recombinant Metabolic Pathway for the Production of 3-HIB in E. coli

For converting the carbon source glycerol to 3-HIB with recombinant E. coli cells, the genes for seven different enzymes were cloned into a series of expression plasmids. Duet-vectors (Merck, Germany) were used for this. This is a system of four expression vectors, which are all compatible with one another and moreover have different antibiotic resistance markers.

In detail, for the conversion of glycerol to 3-HIB, the genes encoding the following enzymes were cloned in expression vectors:

1. Glycerol dehydratase (EC 4.2.1.30) (GD) from Klebsiella pneumoniae. The enzyme catalyses the adenosylcobalamine-dependent dehydration of glycerol to 3-HPA (3-hydroxypropionaldehyde). It consists of 3 subunits (GD-alpha, GD-beta and GD-gamma), which are encoded in K. pneumoniae by 3 genes (gldA, gldB and gldC) in one operon.

2. Reactivation factor from K. pneumoniae. Since adenosylcobalamine-dependent glycerol-dehydratases are inactivated by glycerol, the activity of a reactivation factor is additionally required for the conversion of glycerol to 3-HPA. The reactivation factor for the glycerol dehydratase from K. pneumoniae is encoded by the genes gdrA and gdrB.

3. Aldehyde dehydrogenase AldH from E. coli. For the reaction of 3-HPA to 3-hydroxypropionic acid (3-HP), the E. coli aldH gene was amplified.

4. Propionyl-CoA synthase (Pcs) from Chloroflexus aurantiacus (encoded by the gene pcs). Propionyl-CoA synthase catalyses the conversion of 3-HP to propionyl-CoA. It is a trifunctional enzyme and contains three functional domains. The acyl-CoA synthetase (ACS) domain catalyses the activation of 3-HP to 3-hydroxypropionyl-CoA. This is then followed by dehydration to acrylyl-CoA, catalysed by the enoyl-CoA hydratase (ECH) domain of Pcs. The enoyl-CoA reductase (ECR) domain of Pcs finally catalyses the NADPH-dependent reduction of acrylyl-CoA to propionyl-CoA. For the purpose described, however, this reaction is irrelevant, because here the intermediate acrylyl-CoA is immediately converted further by the next enzyme (crotonyl-CoA carboxylase/reductase, see below).

5. Crotonyl-CoA carboxylase/reductase (enzyme E₁)(Ccr) from Rhodobacter sphaeroides (encoded by the gene ccR). The principal activity of Ccr is the reductive carboxylation of crotonyl-CoA to ethylmalonyl-CoA. However, the enzyme displays broad substrate-specificity and converts acrylyl-CoA to methylmalonyl-CoA very efficiently.

6. Malonyl-CoA reductase (Mcr, E₁₀ and E₁₁) from Sulfolobus tokodaii (encoded by the gene mcr. Mcr preferentially catalyses the NADPH-dependent reduction of malonyl-CoA to malonate-semialdehyde. However, it also displays a subsidiary activity with methylmalonyl-CoA as substrate and converts this to methylmalonate-semialdehyde.

7. 3-Hydroxyisobutyrate dehydrogenase (E₁₂) (3-HIB-DH) from Thermus thermophilus (encoded by the gene MmsB). This enzyme catalyses the NADPH-dependent, reversible reaction of methylmalonate-semialdehyde to 3-hydroxyisobutyric acid (3-HIB).

The cloning strategy for the heterologous overexpression of the enzymes described above is described in detail in the following.

Construction of plasmid pACYCDuet-KpGDRF for overexpression of glycerol dehydratase reactivation factor (GDRF).

First the genes gdrA (synonym ORF4) and gdrB (synonym ORF2b), which encode the two subunits of the K. pneumoniae GDRF, were amplified by PCR. Chromosomal DNA from the strain K. pneumoniae DSM2026 was used as the template.

The following oligonucleotides were used for amplification of gdrA:

orf4fw (5′-TGA AGA TCC TAG GAG GTT TAA ACA TAT GCC GTT AAT AGC CGG GAT TG-3′) and
orf4Salrv (5′-TAT ATA GTC GAC TTA ATT CGC CTG ACC GGC CAG-3′;
SalI recognition sequence underlined).

The following oligonucleotides were used for amplification of gdrB:

orf2bPcifw (5′-TAT ATA ACA TGT CGC TTT CAC CGC CAG GC-3′; PciI recognition sequence underlined) and
orf2brv (5′-CAT ATG TTT AAA CCT CCT AGG ATC TTC AGT TTC TCT CAC TTA ACG GCA GG-3′).

The PCR products obtained were subsequently fused together by crossover-PCR.

For this, the following oligonucleotides were used:

orf2bNcofw (5′-TAT ATA CCA TGG CGC TTT CAC CGC CAG GC-3′;
NcoI recognition sequence underlined) and orf4Salrv (5′-TAT ATA GTC GAC TTA ATT CGC CTG ACC GGC CAG-3′;
Sail recognition sequence underlined)

The PCR product (2220 bp) was purified by means of the QIAquick-PCR-purification kit from Qiagen, Hilden, according to the manufacturer's instructions and ligated into the vector pCR-BluntII-TOPO preserving the pCR-BluntII-Topo-KpGDRF vector. Ligation and subsequent transformation in E. coli cells are carried out according to the instructions of the manufacturer Invitrogen Corporation, Carlsbad (Zero Blunt TOPO PCR Cloning Kit).

The GDRF sequence was then cut out of the vector by digestion of pCR-BluntII-Topo-KpGDRF with PciI and Sail and ligated into the pACYC-Duet expression vector spliced with NcoI and Sail, preserving pACYCDuet-KpGDRF (6142 bp).

Construction of plasmid pAS50_Ec_aldH for overexpression of K. pneumoniae glycerol dehydratase (GD) and E. coli aldehyde dehydrogenase AldH.

The three subunits of K. pneumoniae GD are naturally arranged in an operon (genes gldA, gldB and gldC). They were amplified by PCR, again using chromosomal DNA from K. pneumoniae DSM2026 as template.

The following oligonucleotides were used for the amplification:

KpGDNdefw (5′-TAT ATA CAT ATG AAA AGA TCA AAA CGA TTT GCA GTA CTG G-3′; NdeI recognition sequence underlined) and
KpGDSalrv (5′-TAT ATA GTC GAC TTA GCT TCC TTT ACG CAG CTT ATG C-3′; SalI recognition sequence underlined)

The amplificate was ligated into the vector pCR-BluntII-TOPO, preserving the pCR-BluntII-Topo-KpGD vector. Ligation and subsequent transformation in E. coli cells was carried out according to the instructions of the manufacturer Invitrogen Corporation, Carlsbad (Zero Blunt TOPO PCR Cloning Kit).

The GD-encoding fragment was cut out with XbaI (blunted by Klenow fill in) and NdeI from the vector pCR-BluntII-Topo-KpGD and ligated into a pET-duet expression vector spliced with NdeI and EcoRV, preserving the plasmid pAS50 (8161 bp). Next, the E. coli aldH gene was amplified. For this, chromosomal DNA from E. coli K₁₂ was used as template, using the oligonucleotides 1228_ald_fp (5′-AAAACATATGAATTTTCATCATCTGGCTTACTGG-3′; NdeI recognition sequence underlined) and

1228_ald_rp (5′-AAAACATATGTATATTTCCTTCTTTCAGGCCTCCAGGCTTATCCAGATG-3′; NdeI recognition sequence underlined) as PCR primers. The PCR amplificate was purified on gel and then ligated into the NdeI site of plasmid pAS50 by digesting with NdeI, obtaining the plasmid pAS50_Ec_aldH (9666 bp).

Construction of plasmid pCDFDuet-1_Rs_ccR_Cau_pcs for overexpression of Chloroflexus aurantiacus propionyl-CoA synthase (Pcs) and of Rhodobacter sphaeroides crotonyl-CoA carboxylase/reductase (CCR).

From pTE13 (cf. Example 3) the ccr gene was once again recloned as NcoI/BamHI fragment into the NcoI/BamHI cleavage sites of the plasmid pCDFDuet-1 (Merck, Germany), obtaining the plasmid pCDFDuet-1_Rs_ccr.

Next, the C. aurantiacus pcs gene was amplified by PCR with the oligonucleotides 1228_Cau_pcs_fp(71) (5′-AAAACATATGATCGACACTGCGCCCCTTGC-3′; NdeI recognition sequence underlined) and 1228_Cau_pcs_rp(74) (5′-AAGACGTCCTACCGCTCGCCGGCCGTCC-3′; AatII recognition sequence underlined), using chromosomal DNA from the strain C. aurantiacus OK-70-fl (DSM 636) as template. After purification by gel extraction, the amplificate was ligated by NdeI/AatII digestion into the correspondingly spliced vector pCDFDuet-1_Rs_ccr, obtaining the plasmid pCDFDuet-1_Rs_ccR_Cau_pcs (10472 bp).

Construction of plasmid pCOLADuet_St_mcr_oCg_Tth_HIBDH_oCg for overexpression of Sulfolobus tokodaii malonyl-CoA reductase (Mcr) and Thermus thermophilus 3-hydroxyisobutyrate dehydrogenase (3-HIB-DH)

First, a variant of the S. tokodaii gene mcr adapted to the codon usage of Corynebacterium glutamicum was produced by gene synthesis (St mcr_oCg). The synthesis was carried out at the company GeneArt, Germany, and the artificial gene St_mcr_oCg was prepared in the form of the plasmid pGA4_MMCoAR_ST (SEQ ID No. 5). pGA4_MMCoAR_ST DNA was used as PCR template, for amplifying the artificial gene St_mcr_oCg with the oligonucleotides 1228_MMCoAR_fp (5′-AACCATGGGCCGCACCCTGAAGG-3′; NcoI recognition sequence underlined) and 1228_MMCoAR_rp (5′-AAGGATCCTTACTTTTCGATGTAGCCCTTTTCC-3′; BamHI recognition sequence underlined). After purification by gel extraction, the amplificate was digested with NcoI/BamHI and ligated into the corresponding cleavage sites of the plasmid pCOLADuet_{—}1 (Merck, Germany), obtaining the plasmid pCOLADuet_St_mcr_oCg. A variant of the T. thermophilus gene MmsB (encoding a 3-HIB-DH) adapted to the codon usage of Corynebacterium glutamicum was also prepared by gene synthesis (GeneArt, Germany), namely in the form of the plasmid pGA4_{—}3HIBDH_TT (SEQ ID No. 6). pGA4_{—}3HIBDH_TT was used as PCR template, for amplifying the artificial gene Tth_HIBDH_oCg with the oligonucleotides 1228_Tth_HIBDH_fp (5′-AAAACATATGGAAAAGGTGGCATTCATCG-3′; NdeI recognition sequence underlined) and 1228_Tth_HIBDH_rp (5′-AAAAGATCTTTAGCGGATTTCCACACCGCC-3′; BglII recognition sequence underlined). After gel extraction, the amplificate was spliced with NdeI/BglII and ligated into the NdeI/BglII cleavage sites of the plasmid pCOLADuet_St_mcr_oCg, obtaining the plasmid pCOLADuet_St_mcr_oCg_Tth_HIBDH_oCg (5620 bp). The 4 plasmids pACYCDuet-KpGDRF, pAS50_Ec_aldH, pCDFDuet-1_Rs_ccR_Cau_pcs and pCOLADuet_St_mcr_oCg_Tth_HIBDH_oCg were subsequently co-transformed according to the manufacturer's protocol into commercially available, chemically competent E. coli BL21 (DE3) cells (Merck, Germany). Selection was carried out on LB-agar supplemented with ampicillin (25 μg/ml), chloramphenicol (17 μg/ml), kanamycin (15 μg/ml) and streptomycin (25 μg/ml).

### 6. Induction of the Expression Plasmids in E. coli

The plasmid-bearing E. coli strains described in Example 5 were cultivated in modified M9 medium (6.8 g/l Na₂HPO₄×2H₂O; 3 g/l KH₂PO₄; 0.5 g/l NaCl; 1 g/l NH₄Cl; 1.25 g/l yeast extract; 1% v/v glycerol; 15 mg/l CaCl₂×2H₂O; 250 mg/l MgSO₄×7H₂O; 1% v/v Gibco MEM Vitamin Solution; 41.9 g/l MOPS). The medium was supplemented with ampicillin (25 μg/ml), chloramphenicol (17 μg/ml), kanamycin (15 μg/ml) and streptomycin (25 μg/ml). The complete cultivation (pre-cultures and main cultures) was carried out on a temperature-controlled shaker at 37° C. First, the strains were cultivated overnight in 5 ml of the medium. Then 20 ml of medium, in a 100 ml flask with baffles, was inoculated in the ratio 1:20 with the overnight culture and cultivated further. On reaching OD₆₀₀ of approx. 0.8, 6 μM cobalamine and 1 μM IPTG were added and incubation continued for a further 4 hours. At this point of time, 2.5 ml of cell suspension was taken and stored at −20° C. awaiting analysis.

3-HIB can be detected and quantified by ion chromatography (IC) and conductivity detection. For this, 2.5 ml samples are thawed at room temperature and centrifuged (10 min, 13 200 rpm). The supernatant is purified using a spray filter (pore size 0.44 μm). Measurement is carried out with a Metrohm Compact IC 761 with autosampler. Mobile phase: 8 mM NaOH. Column: Dionex AS15 4×250 mm, precolumn AG15 4×50 mm. Column temperature: 25° C. Flow rate: 1.4 ml/min. Injection volume: 10 μl.

1-18. (canceled)

19. An isolated polynucleotide, which is selected from the group consisting of:
a) a sequence of SEQ ID NO: 1,
b) a sequence that is at least 80% identical to the sequence of SEQ ID NO: 1,
c) a sequence that hybridizes with the antisense strand of SEQ ID NO: 1 at 68° C. in 2×SSC,
d) a derivative of SEQ ID NO: 1 obtained by substitution, addition, inversion and/or deletion of from 1 to 100 bases, wherein the said derivative encodes a protein or peptide,
e) an intron-free sequence, which is obtained from SEQ ID NO: 1,
f) a sequence that is at least 80% identical to the sequence of the intron-free sequence obtained from SEQ ID NO: 1
g) a sequence that hybridizes with the antisense strand of the intron-free sequence obtained from SEQ ID NO: 1 at 68° C. in 2×SSC,
h) a derivative of the antisense strand of the intron-free sequence obtained from SEQ ID NO: 1, which is obtained by substitution, addition, inversion and/or deletion of from 1 to 100 bases, wherein the said derivative encodes a protein or peptide,
i) a sequence that encodes a protein or peptide which comprises the amino acid sequence of SEQ ID NO: 2,
j) a sequence that is at least 80% identical to the sequence that encodes a protein or peptide which comprises the amino acid sequence of SEQ ID NO: 2,
k) a sequence that hybridizes with the antisense strand of the sequence that encodes a protein or peptide which comprises the amino acid sequence of SEQ ID NO: 2 or hybridizes in degeneration of a genetic code,
l) a derivative of the sequence that encodes a protein or peptide which comprises the amino acid sequence of SEQ ID NO: 2, which is obtained by substitution, addition, inversion and/or deletion of from 1 to 100 bases, wherein the said derivative encodes a protein or peptide,
m) a sequence with neutral sense mutations of SEQ ID NO: 1, and
n) a fully complementary sequence to one of from a) to m).

20. A vector comprising the DNA sequence according to claim 19.

21. A method comprising transforming a cell with the vector according to claim 20.

22. A transformed cell, obtained by transformation with the vector according to claim 20.

23. An isolated polypeptide, comprising the amino acid sequence with SEQ ID NO: 2 or an amino acid sequence that is obtained when at most 10 amino acids in SEQ ID NO: 2 are deleted, inserted, substituted or alternatively added onto at least one of the C- and N-terminal end of the amino acid sequence with SEQ ID NO: 2.

24. A cell that is genetically engineered to form more 3-hydroxyisobutyric acid or derivatives thereof in comparison with its wild type.

25. The cell according to claim 24, wherein the cell has, in comparison with its wild type, an increased activity of an enzyme E₁, which is encoded by a polynucleotide selected from the group consisting of:
a) a sequence of SEQ ID NO: 1,
b) a sequence that is at least 80% identical to the sequence of SEQ ID NO: 1,
c) a sequence that hybridizes with the antisense strand of SEQ ID NO: 1 at 68° C. in 2×SSC,
d) a derivative of SEQ ID NO: 1 obtained by substitution, addition, inversion and/or deletion of from 1 to 100 bases, wherein the said derivative encodes a protein or peptide,
e) an intron-free sequence, which is obtained from SEQ ID NO: 1,
f) a sequence that is at least 80% identical to the sequence of the intron-free sequence obtained from SEQ ID NO: 1
g) a sequence that hybridizes with the antisense strand of the intron-free sequence obtained from SEQ ID NO: 1 at 68° C. in 2×SSC,
h) a derivative of the antisense strand of the intron-free sequence obtained from SEQ ID NO: 1, which is obtained by substitution, addition, inversion and/or deletion of from 1 to 100 bases, wherein the said derivative encodes a protein or peptide,
i) a sequence that encodes a protein or peptide which comprises the amino acid sequence of SEQ ID NO: 2,
j) a sequence that is at least 80% identical to the sequence that encodes a protein or peptide which comprises the amino acid sequence of SEQ ID NO: 2,
k) a sequence that hybridizes with the antisense strand of the sequence that encodes a protein or peptide which comprises the amino acid sequence of SEQ ID NO: 2 at 68° C. in 2×SSC,
l) a derivative of the sequence that encodes a protein or peptide which comprises the amino acid sequence of SEQ ID NO: 2, which is obtained by substitution, addition, inversion and/or deletion of from 1 to 100 bases, wherein the said derivative encodes a protein or peptide,
m) a sequence with neutral sense mutations of SEQ ID NO: 1.

26. The cell according to claim 24, wherein the cell comprises an exogenous DNA comprising a polynucleotide selected from the group consisting of:
a) a sequence of SEQ ID NO: 1,
b) a sequence that is at least 80% identical to the sequence of SEQ ID NO: 1,
c) a sequence that hybridizes with the antisense strand of SEQ ID NO: 1 at 68° C. in 2×SSC,
d) a derivative of SEQ ID NO: 1 obtained by substitution, addition, inversion and/or deletion of from 1 to 100 bases, wherein the said derivative encodes a protein or peptide,
e) an intron-free sequence, which is obtained from SEQ ID NO: 1,
f) a sequence that is at least 80% identical to the sequence of the intron-free sequence obtained from SEQ ID NO: 1
g) a sequence that hybridizes with the antisense strand of the intron-free sequence obtained from SEQ ID NO: 1 at 68° C. in 2×SSC,
h) a derivative of the antisense strand of the intron-free sequence obtained from SEQ ID NO: 1, which is obtained by substitution, addition, inversion and/or deletion of from 1 to 100 bases, wherein the said derivative encodes a protein or peptide,
i) a sequence that encodes a protein or peptide which comprises the amino acid sequence of SEQ ID NO: 2,
j) a sequence that is at least 80% identical to the sequence that encodes a protein or peptide which comprises the amino acid sequence of SEQ ID NO: 2,
k) a sequence that hybridizes with the antisense strand of the sequence that encodes a protein or peptide which comprises the amino acid sequence of SEQ ID NO: 2 at 68° C. in 2×SSC,
l) a derivative of the sequence that encodes a protein or peptide which comprises the amino acid sequence of SEQ ID NO: 2, which is obtained by substitution, addition, inversion and/or deletion of from 1 to 100 bases, wherein the said derivative encodes a protein or peptide,
m) a sequence with neutral sense mutations of SEQ ID NO: 1.

27. The cell according to claim 25, wherein the cell has, in comparison with its wild type, an increased activity of an enzyme E₁₁, which is encoded by a polynucleotide selected from the group consisting of:
a) a sequence of SEQ ID NO: 3,
b) a sequence that is at least 80% identical to the sequence of SEQ ID NO: 3,
c) a sequence that hybridizes with the antisense strand of SEQ ID NO: 3 at 68° C. in 2×SSC,
d) a derivative of SEQ ID NO: 3 obtained by substitution, addition, inversion and/or deletion of from 1 to 100 bases, wherein the said derivative encodes a protein or peptide,
e) an intron-free sequence, which is obtained from SEQ ID NO: 3,
f) a sequence that is at least 80% identical to the sequence of the intron-free sequence obtained from SEQ ID NO: 3
g) a sequence that hybridizes with the antisense strand of the intron-free sequence obtained from SEQ ID NO: 3 at 68° C. in 2×SSC,
h) a derivative of the antisense strand of the intron-free sequence obtained from SEQ ID NO: 3, which is obtained by substitution, addition, inversion and/or deletion of from 1 to 100 bases, wherein the said derivative encodes a protein or peptide,
i) a sequence that encodes a protein or peptide which comprises the amino acid sequence of SEQ ID NO: 4,
j) a sequence that is at least 80% identical to the sequence that encodes a protein or peptide which comprises the amino acid sequence of SEQ ID NO: 4,
k) a sequence that hybridizes with the antisense strand of the sequence that encodes a protein or peptide which comprises the amino acid sequence of SEQ ID NO: 4 at 68° C. in 2×SSC,
l) a derivative of the sequence that encodes a protein or peptide which comprises the amino acid sequence of SEQ ID NO: 4, which is obtained by substitution, addition, inversion and/or deletion of from 1 to 100 bases, wherein the said derivative encodes a protein or peptide,
m) a sequence of SEQ ID NO: 3 within the degeneration of the genetic code,
n) a sequence with neutral sense mutations of SEQ ID NO: 3.

28. The cell according to claim 24, wherein the cell is of the genus Rhodobacter sphaeroides.

29. A method of production of a genetically engineered cell, comprising the steps of increasing the activity of the enzyme E₁, which is encoded according to claim 19, or which has the amino acid sequence with SEQ ID NO: 2 or an amino acid sequence that is at least 50% identical to the amino acid sequence according to SEQ ID NO: 2, in a cell.

30. The method according to claim 29, wherein the method further comprises increasing the activity of the enzyme E₁₁, which is encoded by a polynucleotide selected from the group consisting of:
A) a sequence of SEQ ID NO: 3,
B) a sequence that is at least 80% identical to the sequence of SEQ ID NO: 3,
C) a sequence that hybridizes with the antisense strand of SEQ ID NO: 3 at 68° C. in 2×SSC,
D) a derivative of SEQ ID NO: 3 obtained by substitution, addition, inversion and/or deletion of from 1 to 100 bases, wherein the said derivative encodes a protein or peptide,
E) an intron-free sequence, which is obtained from SEQ ID NO: 3,
F) a sequence that is at least 80% identical to the sequence of the intron-free sequence obtained from SEQ ID NO: 3
G) a sequence that hybridizes with the antisense strand of the intron-free sequence obtained from SEQ ID NO: 3 or hybridizes at 68° C. in 2×SSC,
H) a derivative of the antisense strand of the intron-free sequence obtained from SEQ ID NO: 3, which is obtained by substitution, addition, inversion and/or deletion of from 1 to 100 bases, wherein the said derivative encodes a protein or peptide,
I) a sequence that encodes a protein or peptide which comprises the amino acid sequence of SEQ ID NO: 4,
J) a sequence that is at least 80% identical to the sequence that encodes a protein or peptide which comprises the amino acid sequence of SEQ ID NO: 4,
K) a sequence that hybridizes with the antisense strand of the sequence that encodes a protein or peptide which comprises the amino acid sequence of SEQ ID NO: 4 at 68° C. in 2×SSC,
L) a derivative of the sequence that encodes a protein or peptide which comprises the amino acid sequence of SEQ ID NO: 4, which is obtained by substitution, addition, inversion and/or deletion of from 1 to 100 bases, wherein the said derivative encodes a protein or peptide,
M) a sequence of SEQ ID NO: 3 within the degeneration of the genetic code,
N) a sequence with neutral sense mutations of SEQ ID NO: 3
or which has the amino acid sequence with SEQ ID No. 04 or possesses an amino acid sequence that is at least 50% identical to the amino acid sequence according to SEQ ID NO: 4, in a cell.

31. The method according to claim 29, wherein the cell is of the genus Rhodobacter sphaeroides.

32. The method according to claim 29, wherein the increase in activity E₁ or E₁₁ is achieved by increasing the copy number of the genes encoding at least one of the E₁ or E₁₁.

33. A genetically engineered cell, obtained by the method according to claim 29.

34. The cell according to claim 33, wherein, in comparison with its wild type, the cell has an increased copy number of the genes encoding the enzymes E₁ and optionally E₁₁.

35. A method of production of 3-hydroxyisobutyric acid or of derivatives thereof, comprising
bringing the cell according to claim 22 in contact with a culture medium comprising a source of carbon, wherein 3-hydroxyisobutyric acid or a derivative thereof is formed from the carbon source; and
obtaining purified 3-hydroxyisobutyric acid or the derivatives thereof.

36. The method according to claim 35, wherein the derivative is polyhydroxyalkanoates comprising 3-hydroxyisobutyric acid as monomer.

37. The isolated polynucleotide according to claim 19, comprising a sequence that is at least 95% identical to the sequence of SEQ ID NO: 1.
